(19) United States
(12) Patent Application Publication
(10) Pub. No.: US 2017/0027976 A1
(43) Pub. Date: Feb. 2, 2017

(54) NOVEL ADEMETIONINE FORMULATIONS

(71) Applicant: METHYLATION SCIENCES INTERNATIONAL SRL, Christ Church (BB)

(72) Inventors: Dechi GUAN, Vancouver (CA); I. David MACDONALD, Surrey (CA); Melody LEVER, North Vancouver (CA)

(21) Appl. No.: 15/304,164

(22) PCT Filed: Apr. 13, 2015
(86) PCT No.: PCT/IB15/01143

§ 371 Date: Oct. 14, 2016

Related U.S. Application Data
(60) Provisional application No. 61/979,288, filed on Apr. 14, 2014.

(51) Int. Cl. A61K 31/7076; A23L 33/10; A23L 2/52; A23L 7/126; A61K 9/00; A23L 2/39

(52) U.S. Cl. CPC A61K 31/7076; A61K 9/0095; A23L 2/39; A23L 2/52; A23L 7/126; A23L 33/10; A23V 2002/00

(57) ABSTRACT

Provided herein are drinkable or edible compositions comprising ademetionine (“SAM-e” or “S-adenosyl-L-methionine”). Also provided herein are methods for improving the delivery of ademetionine. Compositions and formulations provided herein increase ademetionine appealability. Also provided herein are methods of treating a disease or disorder in a subject by administering drinkable or edible compositions comprising ademetionine.

### CROSS-REFERENCE TO RELATED APPLICATIONS AND CLAIM TO PRIORITY

This application claims the benefit of U.S. Provisional patent application Ser. No. 61/979,288, filed Apr. 14, 2014, which is incorporated herein by reference in its entirety.

### TECHNICAL FIELD

The invention relates to drinkable or edible compositions comprising ademetionine. More particularly, the invention concerns compositions and methods for improving the appealability of ademetionine. The invention is directed to methods of treating a disease or disorder in a subject and/or improving the nutritional status of a subject by administering drinkable or edible compositions comprising ademetionine.

### BACKGROUND OF THE INVENTION

Ademetionine (also referred to as “S-adenosyl-L-methionine”, “SAM” or “SAMe”) is a naturally occurring compound that is present in almost every tissue throughout the body. Aside from water, ademetionine is considered the second most common metabolic molecule-adenosine triphosphate (ATP) being the most common. Ademetionine is available as an over-the-counter dietary supplement in a number of countries and by prescription in Europe. Supplementation with ademetionine has been tested and showed efficacious for the treatment of various ailments, including arthritis, Alzheimer's, liver disease and depression. Unfortunately, however, the uptake of ademetionine is very low (<5%) and therefore large doses are required daily. Thus, there is a need for enhancing the mode of delivery of ademetionine.

### SUMMARY OF THE INVENTION

Drinkable or edible nutritional, pharmaceutical or veterinary products are becoming increasingly popular. In particular, the demand for shelf-stable beverages such as milkshakes or drink crystals, sprinkles, or powders is steadily rising as well as edible products such as snack bars, gels or other foods. Because of this, there is a need in the art for products and processes which improve, for example, the taste, texture and stability of such compositions.

Provided herein are compositions, uses, and methods for enhancing the manufacture and delivery of edible or drinkable ademetionine. Also provided herein are compositions, uses, and methods for enhancing the taste of ademetionine. Also provided herein are compositions, uses, and methods for enhancing the consistency of ademetionine products. Also provided herein are compositions, uses, and methods for enhancing the texture of ademetionine. Also provided herein are compositions, uses, and methods for enhancing the mouthfeel of ademetionine. Also provided herein are compositions, uses, and methods for enhancing the physical stability of ademetionine. Also provided herein are compositions, uses, and methods for enhancing the packaging of ademetionine. Also provided herein are compositions, uses, and methods for enhancing the marketing of ademetionine. Also provided herein are compositions, uses, and methods for enhancing the likelihood of continued use and/or compliance of ademetionine. Also provided herein are compositions, uses, and methods for enhancing the appealability of ademetionine. Also provided herein are compositions, uses, and methods of ingesting or administering ademetionine. In certain embodiments, compositions, uses, and methods described herein provide improved ademetionine levels in vivo as compared to conventional dosage forms of ademetionine.

“Drinkable or edible compositions” as used herein are meant to include drinkable or edible dosage forms comprising ademetionine including solutions, suspensions (e.g. some milkshakes or drinks/beverages), slurries, slushies, smoothies, powders, drink crystals, sprinkles, teas, bubble teas, soups, and medicaments as well as other instant particulate dry mix forms for nutritional, pharmaceutical and/or veterinary use. Instant particulate dry mix forms, including powders and drink crystals, provide a drinkable or edible beverage when mixed with water or other liquid substance. Examples of instant particulate dry mix forms for use in the invention include, but are not limited to, instant soup (including any and all forms of instant soups such as Cup-a-Soup®, ramen noodle soup, those with dried soup stock, those with powdered soup, etc.), instant coffee (including any and all forms of coffee drinks such as regular, decaffeinated, lattes, cappuccinos, americanos, mochas, etc.) “Edible compositions” is also meant to include edible forms such as snack bars (including ‘protein bars’ or ‘meal-replacement bars’), wafers, crackers, cookies, soups, cereals, cereal bars, cereal clusters, granola, chocolate bars, chocolate clusters, chocolate chips, cakes, yogurts, soups, pearls (“boba”) or tapioca balls (such as those found in bubble teas) and other forms that may be ingested and which include ademetionine (i.e. other “foodstuffs”) including, but not limited to, gummies, gummy bears, gummy or chewy products (including carbohydrate and/or energy chews and/or gels), confectionary products, including bakers' confections and sugar confections such as candies, pastilles and sweets. “Chewy” products are meant to include those designed to be ingested as opposed to such non-ingestible chewy items such as chewing gum which may be designed for buccal delivery of agents. Thus in some embodiments edible or drinkable ademetionine compositions are not designed for buccal delivery of ademetionine. In some embodiments edible or drinkable ademetionine compositions may be both edible and drinkable. In some embodiments edible or drinkable ademetionine compositions are gluten-free. In some embodiments edible or drinkable ademetionine compositions are sugar-free. In some embodiments edible or drinkable ademetionine compositions are dairy-free. In some embodiments edible or drinkable ademetionine compositions are suitable for consumption by vegetarian and/or vegan subjects. In some embodiments edible or drinkable ademetionine compositions are suitable for consumption by diabetic subjects.

In some embodiments, compositions described herein are nutritional supplements. In some embodiments, compositions described herein are a medical food. Medical foods are defined by the U.S. Food and Drug Administration as a food which is formulated to be consumed or administered enterally under the supervision of a physician and which is intended for the specific dietary management of a disease or condition for which distinctive nutritional requirements, based on recognized scientific principles, are established by medical evaluation. In some embodiments, compositions described herein are pharmaceutical compositions. In some embodiments, compositions described herein are compositions for veterinary use.

In certain embodiments, compositions, uses, and methods described herein provide improved ademetionine appealability as compared to conventional dosage forms of ademetionine.

“Appealability” as described herein is meant to include anything that makes drinking, eating, ingesting, digesting, packaging, marketing, purchasing or selling ademetionine more appealing than conventional ademetionine compositions, such as an improved taste, texture, viscosity, mouthfeel, smell, presentation, packaging, manufacturing process, storage conditions, shipping conditions, stability, shelf-life, ease of drinking or eating, ease of ingesting, ease of digesting, and/or cost.

“Conventional dosage forms” of ademetionine or “conventional ademetionine compositions” are meant to include tablets and capsules comprising ademetionine including, for example, ademetionine products which are commercially available today.

In some embodiments, provided herein are improved methods of ingesting ademetionine by providing ademetionine in a drinkable or edible composition. Preferably, said method of ingesting ademetionine is faster, easier and/or less frequent than ingesting conventional ademetionine compositions.

In certain embodiments, provided herein are edible or drinkable compositions, uses, and methods comprising ademetionine and one or more gallic acid ester. In some embodiments, a gallic acid ester provided herein is selected from the group consisting of methyl gallate, ethyl gallate, propyl gallate, butyl gallate, isobutyl gallate, isoamyl gallate, octyl gallate, dodecyl gallate, lauryl gallate, hexadecyl gallate, and cetyl gallate. In some embodiments, a gallic acid ester provided herein is ethyl gallate, isoamyl gallate, propyl gallate or octyl gallate. In some embodiments, edible or drinkable compositions, uses, and methods provided herein comprise ademetionine and one or more gallic acid esters as disclosed in International Publication No. WO2014/059522, which is incorporated by reference herein in its entirety.

In some embodiments, provided herein are edible or drinkable compositions comprising ademetionine and one or more gallic acid esters, wherein the ratio (weight:weight) of said gallic acid ester to ademetionine is from 5:1 to 1:400. In some embodiments, the one or more gallic acid ester is selected from ethyl gallate, isoamyl gallate, propyl gallate and/or octyl gallate and the weight ratio of said ethyl gallate, isoamyl gallate, propyl gallate and/or octyl gallate:ademetionine is from 1:1 to 1:2, 1:2 to 1:3, 1:3 to 1:4, 1:4 to 1:5, 1:5 to 1:6, 1:6 to 1:7, 1:7 to 1:8, 1:8 to 1:9, 1:9 to 1:10, 1:10 to 1:11, 1:11 to 1:12, 1:12 to 1:13, 1:13 to 1:14, 1:14 to 1:15, or 1:15 to 1:16.

In some embodiments, provided herein are edible or drinkable compositions comprising ademetionine and one or more gallic acid ester, comprising about 1 to about 200 mg of gallic acid ester. In some embodiments, said formulations comprise about 5 to about 10 mg, about 10 to about 50 mg, about 50 to about 100 mg, about 100 to about 150 mg, about 150 to about 200 mg, about 200 to about 250 mg, about 250 to about 300 mg, about 300 to about 350 mg, about 350 to about 400 mg or greater than about 400 mg gallic acid ester.

In some embodiments, provided herein are edible or drinkable compositions comprising ademetionine and one or more gallic acid ester, wherein said composition comprises 0.1 to 80% by weight gallic acid ester. Other exemplary embodiments comprise from 0.25 to 1%, 1 to 2%, 2 to 3%, 3 to 4%, 4 to 5%, 5 to 10%, 10 to 15%, 15 to 20%, 20 to 25%, 25 to 30%, 30 to 35%, 35 to 40%, 40 to 50%, 50 to 60%, 60 to 70%, 70 to 80%, 80-90% or greater than 90% by weight gallic acid ester. The percentage by weight is based on the weight of the total dosage form.

In some embodiments, provided herein are edible or drinkable compositions comprising ademetionine and one or more gallic acid ester, comprising about 10 to about 3600 mg of ademetionine. When referring to the amount of ademetionine it is intended to mean the ademetionine ion (i.e. the S-adenosylmethionine ion).

In some embodiments, provided herein are edible or drinkable compositions and formulations comprising ademetionine and one or more gallic acid ester, wherein said composition comprises at least 10% by weight ademetionine. In some embodiments, said edible or drinkable compositions or formulations comprise at least 20% by weight ademetionine. In some embodiments, said compositions or formulations comprise at least 30% by weight ademetionine. In some embodiments, said edible or drinkable compositions or formulations comprise at least 40% by weight ademetionine. In some embodiments, said edible or drinkable compositions or formulations comprise at least 50% by weight ademetionine. In some embodiments, said c edible or drinkable compositions or formulations comprise at least 60% by weight ademetionine. In some embodiments, said edible or drinkable compositions or formulations comprise at least 70% by weight ademetionine. In some embodiments, said edible or drinkable compositions or formulations comprise at least 80% by weight ademetionine. In some embodiments, said edible or drinkable compositions or formulations comprise at least 90% by weight ademetionine. In some embodiments, said edible or drinkable compositions or formulations comprise from about 10 to 90% by weight ademetionine. In some embodiments, said edible or drinkable compositions or formulations comprise from about 5 to 10%, 10 to 15%, 15 to 20%, 20 to 25%, 25 to 30%, 30 to 35%, 35 to 40%, 40 to 50%, 50 to 60%, 60 to 70%, 70 to 80%, 80-90% or greater than 90% by weight ademetionine. When referring to the percent by weight of ademetionine it is intended to mean the ademetionine ion.

In some embodiments, said edible or drinkable compositions or formulations comprise from about 10 mg to about 3600 mg ademetionine. In some embodiments, said edible or drinkable compositions or formulations comprise from about 10 mg to about 3600 mg ademetionine or 10 to about 1600 mg ademetionine. In some embodiments, said edible or drinkable compositions or formulations comprise from about 10 to 50 mg, 50 to 100 mg, 100 to 200 mg, 200 to 400 mg, 400 to 600 mg, 600 to 800 mg, 800 to 1000 mg, 1000 to 1200 mg, 1200 to 1400 mg, 1400 to 1600 mg, 1600 to 2000 mg, or 2000 to 3600 mg ademetionine.

In some embodiments, ademetionine is a salt of ademetionine. In specific embodiments, the ademetionine salt is the S-adenosylmethionine disulfate tosylate salt or the S-adenosylmethionine 1,4-butanedisulfonate salt. In other specific embodiments, the ademetionine salt is an indole-3-propionic acid salt of S-adenosylmethionine such as those described in International Patent application WO 2014113609 which is incorporated herein by reference in its entirety. In other specific embodiments, the ademetionine salt is formed with and/or contains at least one of lactose, maltose, dextran, chitosan, phytic acid, inositol, magnesium oxide, and one or more alkali metals.

In some embodiments, provided herein are edible or drinkable compositions which provide increased plasma and/or serum ademetionine levels. In some embodiments, the composition when administered to a selected subject group provides in said selected subject group an average maximum ademetionine blood plasma concentration (average Cₘₐₓ) of at least about 120-300 ng/mL per each 100 mg of ademetionine ion.

In some embodiments, the composition when administered to a selected subject group provides in said selected subject group an average maximum ademetionine blood plasma concentration (average Cₘₐₓ) of at least about 120 ng/mL per each 100 mg of ademetionine ion.

In some embodiments, the composition when administered to a selected subject group provides in said selected subject group an average ademetionine Cₘₐₓ of at least about 12-30 ng/mL per each 10 mg of ademetionine ion.

In other embodiments, the composition when administered to a selected subject group provides in said selected subject group an average ademetionine Cmax of at least about 1.2-3.0 ng/mL per each 1 mg of ademetionine ion.

In some embodiments, provided herein are edible or drinkable compositions which when administered to a selected subject group provides in said selected subject group an average AUC of at least about 800-1000 ng·h/mL per each 100 mg dosage of ademetionine ion.

In some embodiments, the composition when administered to a selected subject group provides in said selected subject group an average ademetionine AUC of at least about 80 ng·h/mL, at least about 85-100 ng·h/mL per each 10 mg of ademetionine ion. In some embodiments, the composition when administered to a selected subject group provides in said selected subject group an average ademetionine AUC of at least about 80 ng·h/mL per each 10 mg of ademetionine ion.

In other embodiments, the composition when administered to a selected subject group provides in said selected subject group an average ademetionine AUC of at least about 8 ng·h/mL, at least about 8.5-10 ng·h/mL per each 1 mg of ademetionine ion. In other embodiments, the composition when administered to a selected subject group provides in said selected subject group an average ademetionine AUC of at least about 8 ng·h/mL per each 1 mg of ademetionine ion.

In some embodiments, the ademetionine ion dose taken is at least 10 mg. In some embodiments, the ademetionine ion dose taken is from 10 to 1600 mg.

In some embodiments, provided herein are edible or drinkable compositions which provide in a selected subject group a lower average Tmax in comparison to a ademetionine control group. In some embodiments, provided herein are compositions which provide in a selected subject group a Tmax or Cmax with reduced variation in comparison to a ademetionine control group. Ademetionine control groups are those wherein the subject or selected subject group is administered the same or similar ademetionine dose with the exception that the ademetionine dose is not administered in a drinkable or edible form of the invention.

In some embodiments, provided herein are edible or drinkable compositions which when administered to a selected subject group provide in said selected subject group an improved ademetionine pharmacokinetic profile such that once a day dosing using compositions described herein is equivalent (or better) to bi-daily dosing of conventional ademetionine compositions that do not contain at least one gallic acid ester or are not in a drinkable or edible dosage form of the invention. “Improved ademetionine pharmacokinetic profile” can be measured by, for example, an equivalent or higher ademetionine AUC or Cmax, a reduced variation of ademetionine Tmax, a reduced side effect profile, and/or an increased rate of onset.

In some embodiments, diseases and/or disorders treatable with ademetionine compositions provided herein are selected from the group consisting of, but not limited to, a mental or psychiatric disorder (e.g. psychotic/mood or non-psychotic mental disorders exemplified by depression and substance related disorders, respectively), a nervous system disease/disorder (e.g. a central nervous system disease exemplified by Alzheimer's), other neurological diseases/disorders (e.g. headaches and sleep disorders), conditions associated with injury to the central nervous system, a liver disease/disorder (e.g. alcoholic liver disease), a cancer (e.g. solid and blood-borne cancers, including those of the gastrointestinal tract), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper- or hypo-methylation, a gastrointestinal disease/disorder, a cardiovascular disease/disorder, atherosclerosis, Lesch-Nyhan disease, a metabolic disease/disorder (e.g. Type 2 diabetes) and a disorder induced in whole or in part by oxidative or free-radical damage. In certain embodiments, the composition comprises ademetionine and at least one gallic acid ester as provided herein. In specific embodiments, the composition comprises ademetionine and ethyl gallate, isoamyl gallate, propyl gallate and/or octyl gallate.

Additional embodiments provided herein relate to drinkable or edible compositions comprising combinations of ademetionine and one or more gallic acid ester along with one or more active ingredients that are commonly prescribed or used for treatment of and/or prophylaxis of various diseases or disorders in a subject. In specific embodiments, the composition comprises ademetionine and one or more of ethyl gallate, isoamyl gallate, propyl gallate and octyl gallate as provided herein.

Also provided herein are methods for treating or preventing and/or prophylaxis in a subject a disease or disorder selected from the group consisting of, but not limited to, a mental or psychiatric disorder (e.g. psychotic/mood or non-psychotic mental disorders exemplified by depression and substance related disorders, respectively), a nervous system disease/disorder (e.g. a central nervous system disease exemplified by Alzheimer's), other neurological disease/disorders (e.g. headaches and sleep disorders), conditions associated with injury to the central nervous system, a liver disease/disorder (e.g. alcoholic liver disease), a cancer (e.g. solid and blood-borne cancers), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper- or hypo-methylation, a gastrointestinal disease/disorder, a cardiovascular disease/disorder, atherosclerosis, Lesch-Nyhan disease, and a disorder induced in whole or in part by oxidative or free-radical damage, comprising drinking or eating a ademetionine composition of the invention, such that said disease is treated or prevented. In specific embodiments, the cancer is a cancer of the gastrointestinal tract, including for example, a bowel cancer, colon cancer, rectal cancer or colorectal cancer. In certain embodiments, the composition comprises ademetionine and one or more of ethyl gallate, isoamyl gallate, propyl gallate and octyl gallate as provided herein. In specific embodiments, the composition comprises ademetionine and ethyl gallate and/or propyl gallate. In a particular embodiment, said disease or disorder is depression. In specific embodiments, said depression is Major depressive disorder (also known as Major depression, Clinical depression), Dysthymic disorder (or also referred to as Dysthymia), Bipolar disorder (formerly referred to as Manic depression), Postpartum depression, Seasonal Affective Disorder (SAD), Anxiety depression, Atypical depression, Melancholic depression, Catatonic depression and Situational depression, Reactive depression, Late-Life depression (and the like), Parkinson's depression, HIV-associated depression, brief recurrent depression, Mild depression, Minor depression, Treatment-Resistant depression (TRD), co-morbid depression, or depression NOS (Not Otherwise Specified).

In some embodiments, any of the drinkable or edible compositions provided herein is used in the treatment of the diseases and disorders described herein.

Also provided herein are methods for administering a drinkable or edible composition of the invention comprising ademetionine wherein said method comprises administering said composition to a patient, subject and/or selected subject group that have fasted prior to administration of said composition. “Fasted” typically is meant to be an overnight fast such that patients (or subjects) are administered the composition at least one hour prior to their first meal of the day (i.e. typically breakfast). In some embodiments, “fasted” conditions are such that subjects begin fasting at least 10 or 12 hours before drug administration and fasting continues for 1 or 4 hours following drug administration. Also provided herein are methods for administering a drinkable or edible composition of the invention comprising ademetionine wherein said method comprises administering said composition to a patient or selected subject group under fed conditions. “Fed” conditions are typically such that the patients/subjects ingest a meal approximately 1-2 hours before being administered the composition of the invention. In some aspects of the invention, under “fed” conditions, subjects start fasting approximately 12 hours before morning breakfast and then receive a meal (often a standardized high-fat, high-calorie meal) approximately 30 minutes before drug administration.

Also provided herein are methods for improving the appealability of ademetionine, wherein said method comprises administering to a subject an exemplary edible or drinkable composition which provides a physiologically effective amount of ademetionine in a composition of the invention. In certain embodiments, said ademetionine composition comprises one or more gallic acid ester. In specific embodiments, said one or more gallic acid ester is selected from the group consisting of ethyl gallate, isoamyl gallate, propyl gallate and octyl gallate.

Further provided herein is a method of making a drinkable or edible composition of ademetionine, wherein said method comprises mixing ademetionine and one or more gallic acid ester and formulating them into an edible or drinkable composition of the invention. In certain embodiments, said one or more gallic acid ester is selected from ethyl gallate, isoamyl gallate, propyl gallate and octyl gallate. Thus in some embodiments, provided herein is a method for improving the appealability of ademetionine, wherein said method comprises administering to a subject an exemplary edible or drinkable composition which provides a physiologically effective amount of ademetionine and at least one gallic acid ester. Also provided in some embodiments is a method of making a formulation for improved appealability of ademetionine, wherein said method comprises combining ademetionine and at least one gallic acid ester and formulating them into a drinkable or edible suspension, powder, milkshake, drink crystal, or sprinkle with or without additional excipients. In other embodiments, provided is a method of administering an edible or drinkable composition comprising ademetionine and ethyl gallate, isoamyl gallate, propyl gallate and/or octyl gallate wherein said method comprises administering said edible or drinkable composition to a patient that has fasted prior to administration of said drinkable or edible composition.

The details of one or more embodiments are set forth in the description below. Other features, objects and advantages will be apparent from the description and the claims. In addition, the disclosures of all patents and patent applications referenced herein are incorporated by reference in their entirety.

### BRIEF DESCRIPTION OF THE FIGURES

FIG. 1 is a graph of the average maximum Ademetionine plasma concentration (Cmax) of beagles who were fed a single 400 mg Ademetionine ion dose from one of ten different oral formulations: (1) a commercially available oral formulation of Ademetionine, (2) MSI Ademetionine formulation with no propyl gallate (“Control Ademetionine”); (3) MSI Ademetionine formulation co-administered with a separate 25 mg propyl gallate formulation (“Control Ademetionine with Separate PG”); (4) MSI Ademetionine formulation co-formulated with 25 mg methyl gallate; (5) MSI Ademetionine formulation co-formulated with 25 mg ethyl gallate; (6) MSI Ademetionine formulation co-formulated with 25 mg propyl gallate; (7) MSI Ademetionine formulation co-formulated with 25 mg butyl gallate; (8) MSI Ademetionine formulation co-formulated with 25 mg isobutyl gallate; (9) MSI Ademetionine formulation co-formulated with 25 mg isoamyl gallate; or (10) MSI Ademetionine formulation co-formulated with 25 mg octyl gallate.

### DETAILED DESCRIPTION OF THE INVENTION

The present investigators have surprisingly discovered that the appealability of ademetionine can be significantly improved when manufactured and administered as a drinkable or edible nutritional, dietary, veterinary and/or pharmaceutical product. Thus some embodiments relate to edible or drinkable compositions, uses, and methods for enhancing the smell, taste, texture, consistency or mouthfeel of ademetionine. Also provided are edible or drinkable compositions, uses, and methods for enhancing the packaging and/or marketing of ademetionine. Also provided herein are edible or drinkable compositions, uses, and methods for enhancing the likelihood of continued use or compliance of use of ademetionine. Also provided herein are edible or drinkable compositions, uses, and methods for enhancing the physical stability, including shelf-life, of ademetionine. In certain embodiments, edible or drinkable compositions, uses, and methods described herein provide improved ademetionine pharmacokinetics in vivo as compared to conventional dosage forms of ademetionine.

“Drinkable or edible compositions” or “edible or drinkable” as used herein is meant to include drinkable and/or edible forms comprising ademetionine including suspensions (e.g. some milkshakes or drinks/beverages), solutions, slurries, slushies, smoothies, powders, drink crystals, sprinkles, and medicaments as well as instant particulate dry mix forms for nutritional, pharmaceutical and/or veterinary use. Instant particulate dry mix forms, including powders and drink crystals, provide a drinkable or edible beverage when mixed with water or other liquid substance. Dry, rapidly-dissolving and room-temperature stable drink powders or crystals of ademetionine is a desirable way to administer, package and market ademetionine products. “Edible compositions” is also meant to include edible forms that comprise ademetionine such as snack bars (including ‘protein bars’ or ‘meal-replacement bars’), wafers, crackers, cookies, soups, cereals, cereal bars, cereal clusters, granola, cakes, yogurts, other baked goods and other forms that may be ingested and which include ademetionine, including gummies or other chew products and confectionary bakers' products or sugar confections. In some embodiments, edible or drinkable ademetionine compositions comprise ademetionine added to edible or drinkable products currently on the market such as, but not limited to, Boost® nutritional food and/or drinks, Ensure® drinks, Carnation Breakfast Essentials® food and/or drinks, Danone® food and/or drinks and Nesquik® food and/or drinks. In some embodiments edible or drinkable ademetionine compositions are gluten-free. In some embodiments edible or drinkable ademetionine compositions are sugar-free. In some embodiments edible or drinkable ademetionine compositions are dairy-free. In some embodiments edible or drinkable ademetionine compositions are suitable for consumption by vegetarian and/or vegan subjects. Thus in some embodiments, compositions described herein are nutritional supplements. In some embodiments, compositions described herein are dietary supplements. In some embodiments, compositions described herein are a medical food. In some embodiments, compositions described herein are pharmaceutical compositions. In some embodiments, compositions described herein are compositions for veterinary use.

“Appealability” as described herein is meant to include any non-tablet or non-capsule form that makes drinking, eating, ingesting, digesting, packaging, marketing, purchasing or selling ademetionine more appealing than conventional ademetionine compositions, such as an improved taste, texture, viscosity, mouthfeel, smell, presentation, packaging, manufacturing process, storage conditions, shipping conditions, stability, shelf-life, ease of drinking or eating, ease of ingesting, ease of digesting, and/or cost.

Other embodiments relate to methods that improve the appealability of ademetionine, e.g. for the treatment of various diseases or disorders in a subject and/or improving the nutritional status of a subject. Additional embodiments relate to combinations of ademetionine and one or more gallic acid ester with one or more active ingredients that are commonly prescribed or used for treatment of and/or prophylaxis of various diseases or disorders in a subject.

As used herein the term “ademetionine” refers to S-adenosyl-L-methionine (or, more simply, “SAM”, “SAM-e” or “SAMe”) including all of the various ademetionine salts. When referring to dose or percentage, the amount (typically in mg) refers to the dose of ademetionine ion. As mentioned above, ademetionine is most commonly available as a stable salt form, e.g. with p-toluenesulfonic acid (see U.S. Pat. No. 3,893,999, incorporated herein by reference in its entirety). Other stable ademetionine salts are described in, for example, U.S. Pat. No. 5,128,249, which describes particular stable salts of ademetionine. Various morphologies of ademetionine are suitable for use in certain embodiments provided herein. Thus, as used herein “ademetionine” refers to the stable salts, amorphous forms, semicrystalline forms and crystalline forms of ademetionine as well as to the ionic form of ademetionine when present in vivo. Amorphous forms of ademetionine can be employed at any particle size and particle size distribution. It is understood that the particle size of individual ademetionine compositions will be tailored to accommodate the given mode of ingestion. For example, the particle size of drinkable composition components including milkshakes, drink crystals and powders, may be smaller than those of edible components. The particle size of drinkable or edible components can largely affect the rate of digestion and consequently other metabolic effects. Particle size can affect the taste, appearance, stability, processability, and functionality of the final ademetionine product. For example, emulsions typically contain essential oils, emulsifiers, and stabilizers in an aqueous continuous phase. The particle size of the emulsions droplet is critical for stability. Given the sub-micron size range of most emulsions, the size analysis should be performed with either laser diffraction or dynamic light scattering (DLS). Ademetionine may also be solubilized or in solution.

In some embodiments, provided are edible or drinkable compositions comprising particles having substantially no particles larger than about 5 mm. “Substantially no particles larger than about 5 mm” means that at least about 95%, more specifically at least about 98% of all particles in the composition are smaller than 5 mm. In some embodiments, provided are edible or drinkable compositions comprising particles having substantially no particles larger than about 5000 microns, 4000 microns, 3000 microns, 2000 microns, 1000 microns, 800 microns, 700 microns, 600 microns, 500 microns, 400 microns, 300 microns, 200 microns, 100 microns, 50 microns, 20 microns or 10 microns. The particle size may be measured by laser diffraction or DLS. In some embodiments, provided are edible or drinkable compositions comprising particles having particle sizes such that 40%, 50%, 60%, 70% or 80% would pass through a sieve which has openings of 800 to 1000 microns, 600 to 800 microns, 400 to 600 microns, 300 to 500 microns, 200 to 300 microns, 100-200 microns, 50-100, or 10-50 microns.

In some embodiments, edible or drinkable ademetionine compositions comprise nano delivery systems including, but not limited to, liposomes, nanoparticles, lipid-stabilized emulsions and micelles. In some embodiments, said nano delivery systems have an average diameter of from 50 to 250 nm or from 80 to 200 nm. In certain embodiments, said nano delivery systems comprising ademetionine act to stabilize ademetionine. In other embodiments, said nano delivery systems encapsulating ademetionine are formulated into a beverage such as a bubble tea. It is thought that the ademetionine may be encapsulated within the ‘bubbles’ of the bubble tea, for example within a polymer or starch coating that is rolled to form small bubbles (or pearls/balls) of which the ademetionine is encapsulated. In specific embodiments, said nano delivery systems comprise ademetionine and at least one gallic acid ester. In some embodiments, said gallic acid ester is selected from ethyl gallate, isoamyl gallate, octyl gallate and propyl gallate.

It is understood that the ademetionine at any particle size or in solubilized form or in solution will be formulated such that the ademetionine is stabilized or protected from degradation such as that caused by water, air, oxygen, temperature, light, pH changes (for example in the stomach), or other factors/agents which case ademetionine to degrade. For example, this “protected ademetionine” or “stabilized ademetionine” may be dry granules or powders which are coated with a coating that protects from moisture (such as PVA-based coatings), or protects from air, or protects from oxygen and/or protects from pH changes (such as enteric coatings); or may be encapsulated ademetionine within a polymer shield that protects from moisture, air, oxygen and/or pH changes; or may be polymer-conjugated ademetionine such that degradation of the ademetionine is slowed; or may be ademetionine formulated in nano delivery systems such that degradation of the ademetionine is slowed. In some embodiments, “stabilized ademetionine” is meant to include those formulations which maintain the integrity of ademetionine at room temperature. Ademetionine integrity may be measured by measuring standard, known degradation products of ademetionine and/or the level of one or more ademetionine isomers, and/or the amount of water. “Stabilized ademetionine” may also be granulates such as those described in U.S. Pat. No. 7,048,948 which is incorporated herein by reference in its entirety.

Formulations for oral administration of ademetionine are typically provided in the art as capsules or tablets, and generally consist of a core “matrix material” as well as one or more coatings; in some cases ademetionine has been provided as a chewing gum. Edible or drinkable “product” or “dosage form” or “composition” as used herein refers to any solid, semi-solid, semi-liquid, liquid or frozen, partially-frozen composition used for oral administration and is exemplified by suspensions (including shelf-ready and frozen milkshakes or drinks), powders, crystals, sprinkles, or other drinkable and/or edible formulations that are not capsules, tablets or gum.

In certain embodiments, a drinkable or edible ademetionine composition disclosed herein is not a hard, soft and/or gel capsule. In certain other embodiments, a drinkable or edible ademetionine composition disclosed herein is not a tablet. In yet other embodiments, a drinkable or edible ademetionine composition disclosed herein is not a minitablet. It is understood that “tablets” are meant to be those of tablet dosage forms of the art which generally are in the range of 50 mg up to 1.5 gram. Minitablets or mini-tablets are known in the art to be around 5 to 50 mg and typically 1 to 3 mm. It is understood that compositions of the invention are not minitablets or mini-tablets. It is also understood that compositions of the invention in some embodiments comprise particles which are less than 5 mg. It is also understood that in certain embodiments, compositions of the invention comprise particles that are from 10 to 5000 microns in diameter. In certain embodiments, a drinkable or edible ademetionine composition disclosed herein is not a chewing gum (i.e. a gum that is not edible, or not recommended to be swallowed/ingested). In certain embodiments, a drinkable or edible ademetionine composition disclosed herein is not an orodispersible tablet. In certain embodiments, a drinkable or edible ademetionine composition disclosed herein is not a chewable tablet for buccal delivery. In some embodiments, a drinkable or edible ademetionine composition disclosed herein does not contain one or more aerobic proteins. In some embodiments, a drinkable or edible ademetionine composition disclosed herein does not contain ten or more added amino acids. In some embodiments, a drinkable or edible ademetionine composition disclosed herein does not contain ten or more added enzymes. In some embodiments, a drinkable or edible ademetionine composition disclosed herein does not contain dissolved oxygen. In some embodiments, a drinkable or edible ademetionine composition disclosed herein does not contain one or more added electrolytes. In some embodiments, a drinkable or edible ademetionine composition disclosed herein does not contain CellFood™. Drinkable and edible ademetionine compositions disclosed herein may be administered using a clinical, pharmaceutical or veterinary dosing regimen. Drinkable and edible ademetionine compositions disclosed herein may also be provided as dietary or nutritional supplements or as a medical food.

In some embodiments, a drinkable or edible composition disclosed herein is a beverage; and in more specific embodiments, a pop, soda, instant drink, alcohol-containing drink, bubble tea, a coffee beverage, smoothie or milkshake. In some embodiments, a drinkable or edible composition disclosed herein is a food or beverage that may be stored and/or sold in a vending machine. In some embodiments, a drinkable or edible composition disclosed herein comprises dissolved carbon dioxide. In some embodiments, a drinkable or edible composition disclosed herein comprises dissolved nitrogen. In some embodiments, a drinkable or edible composition disclosed herein is a food or beverage that may be stored and/or sold in a grocery or convenience store. In some embodiments, a drinkable or edible composition disclosed herein is a food or beverage that may be stored and/or sold in a restaurant, bistro, café, concession stand, liquor store, food truck and/or food cart. In some embodiments, a drinkable or edible composition disclosed herein is a food or beverage that may be purchased and/or sold online (i.e. through an internet provider). A “smoothie” is a blended, usually chilled, sweet beverage made from fresh fruit. It is sometimes blended with crushed ice, frozen fruit, or frozen yogurt. Smoothies have a milkshake-like consistency which is thicker than slush drinks, but unlike milkshakes, they do not usually contain cow's milk or ice cream. Smoothies are marketed to health-conscious people, and some restaurants offer add-ins such as soy milk, whey powder, green tea, herbal supplements, or nutritional supplement mixes. A “soda” or “pop” is meant to include any carbonated drink, or any drink which contains dissolved carbon dioxide. A “marble soda” or “marble pop” is meant to include Ramune™ and Marble Pop™ and other such drinks present in a ramune bottle or similar Codd-neck bottle (or marble-in-the-neck bottle) which is different than conventional soda and/or pop bottles and/or cans. Codd-neck bottles were originally designed in the 1800s and protected by a number of patents including the 1878 US patent number 8,372 which is incorporated herein by reference in its entirety. “Instant drinks” are meant to include anything that has ademetionine in a non-wet form, such as freeze-dried, dried, powder, lyophilized, crystallized or other dry material such that water or other liquid is absent or minimal yet may be added prior to drinking. Ademetionine is preferably mixed with hot and not boiling liquid so as to minimize racemization of the ademetionine. In some embodiments a label is added to the edible or drinkable ademetionine composition with instructions to add hot or warm, and not boiling, liquid (water, for example).

It is well known in these arts that ademetionine is highly hygroscopic. Thus in some embodiments it is desirable to protect the ademetionine from water or moisture until just prior to eating and/or drinking. Thus a multi-chamber or multi-compartment bottle, container or can is preferred for drinkable ademetionine compositions. Within one embodiment a multi-chamber (e.g., a two-chamber) vial is provided with a first chamber having a dry component (e.g., one of the ademetionine compositions provided herein), and a second chamber having a wet component. Within various embodiments the first chamber can be placed under a vacuum (and/or the second chamber may also be placed under a vacuum. Within other embodiments the first and or second chamber may be filled with a gas (e.g., nitrogen or carbon dioxide). One representative example of a multi-chamber device is a Mix-O-Vial (see e.g., U.S. Publication No. US 2004/0039366, which is hereby incorporated by reference in its entirety. Within other embodiments, a bottle or delivery device may be modified to have two chambers. For example, a ramune bottle or Codd-bottle (as described in more detail below) can be modified to have an upper chamber where an ademetionine composition as described herein is stored (above the marble), and not released until just prior to drinking (when the marble is released). The ramune or Codd-bottle can be modified to make a multi-chamber bottle with by manufacturing the bottle with two chambers, or, by manufacturing a sealable lid above the marble component which can contain the ademetionine composition. A multi-chamber or multi-compartment container is also preferred for edible ademetionine compositions, including for example 2-compartment containers such as those that would typically comprise a yogurt and a granola in separate compartment which may be mixed prior to eating as exemplified in US patent application number 2011/0303678 which is incorporated herein by reference in its entirety. Also preferred is a multi-compartment, one piece container such as those exemplified in US patent application number 2015/004283 and U.S. Pat. Nos. 7,537,131 and 8,866,056 which are incorporated herein by reference in their entirety. In some preferred embodiments, a multi-compartment container is designed with separate, detachable compartments which may or may not be manufactured and/or packaged together such as those exemplified in US patent application number 2014/0079849 which is incorporated herein by reference in its entirety.

A drinkable ademetionine composition is a desirable way to consume large quantities of ademetionine that may be needed daily. Even more desirable are drinks such as Ramune™ or Marble Soda™ beverages. Thus in certain embodiments provided are compositions comprising ademetionine which is included in a ramune bottle or marble soda or Codd-neck bottle. Specific examples of these bottles for use in the invention also comprise unique caps for opening the bottles, such as a marble soda drink cap with temper-proof design that looks like a hypostyle annulet with several easy breaking columns. Each column from underneath the hypostyle is linked to the annulet. It sets several small plates between each column and handholds around the annulet. This opener security combination can be capped into the top of the bottle easily and downward anchored by the plates. It can achieve the security function for the consumer to prevent other people from willfully opening the bottle head. If the easy break points break, it can be told that the drink has been opened. Moreover, the consumer picks up the opener from the easy break point and reverse the bottle, then use the traditional way to open and drink the marble soda drink.

Other desirable drinkable ademetionine compositions include bubble teas. Bubble tea, also known as boba tea or pearl milk tea, contains a tea drink, typically milky, which has small tapioca or jelly balls (also called “pearls”) that sink to the bottom. These can be shaken and mixed prior to drinking. Bubble tea can also include fruit or fruit jellies to add to the taste and appeal of the drink. The pearls in bubble tea are well known in the art and often made by consumers at home. They are most often made of tapioca starch which has been mixed with boiling water to form a paste which can then be rolled into small balls or pearls that are allowed to dry. These pearls absorb liquid (e.g. water in the tea) upon addition to the tea and soften over a short period of time. In certain aspects of the invention, ademetionine-containing pearls are made using a polymer or hydrogel which can protect the ademetionine from moisture or other external ingredients or factors. The polymer or hydrogel may itself form the pearl or may be used to coat a pre-made pearl-containing ademetionine.

Other desirable drinkable ademetionine compositions include room-temperature, shelf-ready milkshake beverages as well as frozen or refrigerated milkshakes. Conventional processes for producing shelf-stable milkshake compositions involve the steps of creating a batch made up of the various compositional components, which typically includes modified food starch; subjecting the batch to high temperature short time (“HTST”) pasteurization, or the like; homogenizing the resulting pasteurized composition; filling the homogenized, pasteurized composition into the requisite containers; and subjecting the filled containers to a retorting treatment. As ademetionine is temperature sensitive it is understood that ademetionine for consumption will be added to the other milkshake ingredients after they have been homogenized and pasteurized and prior to filling into the requisite containers. The quality of milkshake compositions prepared by such conventional processes is highly dependent on processing conditions and a particular composition may vary in consistency with respect to mouthfeel and physical stability depending on how the composition is made, even though all of the ingredients in the composition and their relative proportions are the same. The majority of these inconsistencies are due to the shearing effects of homogenization on swollen starch granules within the composition. During the standard pasteurization step, starch granules swell and the pressure of homogenization sheers these larger granules and greatly affects the texture and consistency of the milkshake. Thus, more preferably, provided herein are ademetionine milkshake compositions which have enhanced texture and consistency. Even more preferably, provided herein are milkshake compositions comprising ademetionine and one or more gallic acid ester. Most preferably, said compositions have enhanced taste, texture and/or consistency.

An edible ademetionine composition is also a desirable way to consume large quantities of ademetionine that may be needed daily. Preferably, said edible composition is a gummy or chewy product.

In certain embodiments, the gummie or chewy product is produced using an open pan method for cooking using a stirred steamed-jacketed confectionery boiling pan. Water is heated to boiling (100° C.) and oxidized starch is added (typically about 5-15% by weight), preferably as a dry mix along with gum arabic (typically about 5-15% by weight). The mixture is placed in a high speed mixer to adequately dissolve the solid products. The oxidized starch gelatinizes on dispersion in excess hot water and then the remaining products are added (for example, fat). This solution is brought to boiling, preferably in the range of 115-125° C., depending on the desired final texture. The hot solution is cooled to a temperature safe for ademetionine, ademetionine is added and the complete solution is then poured into moulds or casts of desired shape (for example a small circular or square shape) and left to cool. In some embodiments, a method of making gummy or chewy ademetionine compositions is provided which method comprises heating oxidized starch until it is gelatinous, mixing the gelatinous starch with a sugar and/or corn syrup, heating/cooking the mixture to at least 110° C., cooling the mixture and then adding ademetionine, and then pouring the mixture into moulds/casts of desired shape and allowing to completely cool to form the gummie or chewy product. There are numerous well-known methods in the art for making various sized and shaped gummy substances such as those described in U.S. Pat. No. 5,560,949 which is incorporated herein by reference in its entirety. As ademetionine is temperature sensitive it is understood that ademetionine for gummy products will be added to the other ingredients after they have been heated to the necessary temperature(s) and prior to filling into the specific shaped molds/casts.

In some embodiments, provided is a composition comprising ademetionine and gum arabic. In some embodiments, provided is a composition comprising ademetionine, a gallic acid ester and gum arabic. In some embodiments, provided is a composition comprising ademetionine and gelatin. In some embodiments, provided is a composition comprising ademetionine, a gallic acid ester and gelatin. In certain embodiments, provided is a composition comprising ademetionine and chicle. In certain other embodiments provided is a composition comprising ademetionine, chicle and a gallic acid ester. In yet other embodiments provided is a composition comprising ademetionine and a butadiene-based synthetic rubber. In some embodiments, provided is an ademetionine composition which does not comprise a gum base. In some embodiments, provided is an ademetionine composition which does not comprise Mint Flavor. In some embodiments, provided is an ademetionine composition which does not comprise F-Melt®. In some embodiments, provided is an ademetionine composition which does not comprise magnesium hydroxide. In some embodiments, provided is an ademetionine composition which does not comprise brewer's dried yeast. In some embodiments, provided is an ademetionine composition which is not Denosyl™. In some embodiments, provided is an ademetionine composition which does not comprise stabilized rice bran. In some embodiments, provided is an ademetionine composition which does not comprise milk thistle. In some embodiments, provided is an ademetionine composition which does not comprise silybin-phosphatidylcholine. In some other embodiments, provided is an ademetionine composition which does not comprise calcium propionate.

In certain embodiments, the edible ademetionine product is a granola product. Granola products are meant to include those which comprise oats/rolled oats and other ingredients. In specific embodiments, the granola product is a granola cluster or flake such as those used in cereals. In more specific embodiments, the edible ademetionine product is a chewy granola product. Chewy granola products are well-known in the art. For example, U.S. Pat. No. 4,451,488 teaches the composition and manufacture of chewy granola products and is hereby incorporated by reference in its entirety. In certain aspects of the invention, cold form sheeting such as that describe in U.S. patent application number 2014/0272009, which is incorporated herein by reference in its entirely, can be used to make the chewy granola product as well a chewy granola base product. Typical chewy granola base products are made from dry particulates, one or more binders, and may optionally include one or more enhancements, toppings, or coatings. Additionally, chewy granola base products may also include additional optional components such as additional grains, fruits, nuts, seeds, fats, proteins, candy, carbonated candy, caffeine or other well-known additives or nutritional supplements so as to enhance specific properties such as taste, nutritional value, or marketability of the chewy granola product.

A difficulty with ademetionine is that it must not be exposed to moisture during processing or storage. Otherwise, the ademetionine will degrade. Hence, it is important that prior to preparing the chewy bar, the ademetionine has, or is coated with, a moisture impervious coating to ensure that premature degradation does not occur during processing. During consumption (chewing), the coating is broken, exposing the ademetionine therein.

Suitable coatings include, but are not limited to moisture barriers such as OpaDry®II as well as enteric coatings, time-release coatings and pH-dependent coatings.

In certain embodiments, the edible ademetionine product is a yogurt or yogurt-containing product. Yogurt or yogurt-containing products are meant to include those which are stored frozen or stored in a refrigerator including but not limited to yogurts which are typically in packaging such that they are eaten with as a spoon (such as the Chobani Flip™ containers or Activia® Parfait Crunch™) or without a utensil (such as yogurt tubes, yogurt pops, and squishable or drinkable yogurts such as Yop® or Danimals®). In specific embodiments, the edible ademetionine product is a yogurt or yogurt-containing product which also comprises granola and/or one or more fruits. In other specific embodiments, the edible ademetionine composition comprises a yogurt or yogurt-containing product in one compartment and an ademetionine-containing topping in a second compartment. The topping may be a fruit mixture, nut mixture, chocolate or other desirable ingredients which are mixed with stabilized ademetionine. Multi-compartment yogurt containers and methods of their manufacture above are well known in these arts such as those described in US 2011/0303678, Typically these containers (often called “parfait” cups) provide a yogurt in one compartment and a topping (such as, for example, granola) in a separate compartment. Toppings may also include vitamins, minerals, probiotics or other nutritional substances. In some aspects of the invention, ademetionine granola compositions as described above are included in such multi-compartment yogurt containers. In other aspects, ademetionine edible or drinkable compositions which are not granola (including, but not limited to, granules, particles, powders, drink crystals, and sprinkles, for example) are included in such multi-compartment yogurt containers such that they are physically separated from the wet yogurt yet may be mixed prior to consumption if desired. In yet other embodiments, a dried yogurt product is combined with stabilized ademetionine.

In some embodiments, edible and drinkable compositions disclosed herein comprise gallic acid esters selected from the group consisting of but not limited to methyl gallate, ethyl gallate, propyl gallate, isopropyl gallate, butyl gallate, isobutyl gallate, amyl gallate, isoamyl gallate, hexyl gallate, isohexyl gallate, heptyl gallate, isoheptyl gallate, octyl gallate, isooctyl gallate, nonyl gallate, isononyl gallate, decyl isodecyl, undecyl gallate, isoundecyl gallate, dodecyl gallate (lauryl gallate), isododecyl gallate, tridecyl gallate, isotridecyl, tetradecyl gallate, isotetradecyl gallate, pentadecyl gallate, isopentadecyl gallate, hexadecyl gallate (cetyl gallate), isohexadecyl gallate, heptadecyl gallate, isoheptadecyl gallate, octadecyl gallate, isoctadecyl gallate, cis-9-hexadecenyl (palmitoleyl) gallate, cis-9-octadecenyl (oleyl) gallate, cis,cis-9,12 octadecadienyl (linoleyl) gallate, trans,trans-9,12-octadecadienyl (linolelaidyl) gallate, cis,cis,cis-9,12,15-octadecatrienyl (linolenyl) gallate, trans,trans,trans-9,12,15-octadecatrienyl (linolenelaidyl) gallate, cis,cis,cis-6,9,12-octadecatrienyl (gamma-linolenyl) gallate, trans 9-octadecenyl (elaidyl) gallate, trans-9-hexadecenyl (palmitelaidyl) gallate, 2-ethylhexyl gallate, 2-hydroxyethyl gallate, 6-O-galloylglucose, hamamelitannin, methoxyethoxyethoxyethyl m-digallate, theaflavin monogallate A & B, and theaflavin digallate. In certain embodiments, the gallic acid ester is not epigallocatechin. In certain specific embodiments, the gallic acid ester is not epigallocatechin gallate. In certain specific embodiments, the gallic acid ester is selected from ethyl gallate, isoamyl gallate, propyl gallate and octyl gallate. In some embodiments, the gallic acid ester is considered a GRAS (Generally Recognized As Safe) substance by the U.S. Food and Drug Administration (FDA). In some embodiments, the gallic acid ester has received a Novel Food approval by either the European Food Safety Authority (EFSA) or the European Medicines Agency (EMA). In a specific embodiment, the gallic acid ester is selected from ethyl gallate and propyl gallate. Thus provided herein are drinkable or edible compositions comprising ademetionine and ethyl gallate and/or propyl gallate. In certain embodiments, drinkable or edible ademetionine compositions disclosed herein are not in the form of a capsule. In certain other embodiments, drinkable or edible ademetionine compositions disclosed herein are not in the form of a tablet. In some embodiments, drinkable or edible ademetionine compositions disclosed herein do not contain one or more aerobic proteins or ten or more amino acids and/or ten or more enzymes.

In some embodiments, compositions disclosed herein are taken such that the daily amount of ademetionine and/or gallic acid ester dosed does not exceed the acceptable daily intake (“ADI”) as established by the Joint FAO/WHO Expert Committee on Food Additives (JECFA).

In some embodiments, drinkable or edible compositions disclosed herein comprising ademetionine and gallic acid ester comprises from 0.25 to 1%, 1 to 2%, 2 to 3%, 3 to 4%, 4 to 5%, 5 to 6% or 6 to 7% by weight gallic acid ester wherein the weight percentage is based on the weight of the total dosage form. In some other exemplary embodiments, said composition comprising ademetionine and a gallic acid ester comprises 7 to 10%, 10 to 15%, 15 to 20%, 20 to 25%, 25 to 30%, 30 to 35%, 35 to 40%, 40 to 50%, 50 to 60%, 60 to 70%, 70 to 80%, 80 to 90% or greater than 90% by weight gallic acid ester.

Some exemplary embodiments relate to “low-dose” drinkable or edible ademetionine compositions. By increasing the bioavailability of ademetionine in the presence of a gallic acid ester, the daily drinkable or edible dose of ademetionine may be substantially lowered by administration of compositions with improved ademetionine uptake in comparison to those formulations that do not contain at least one gallic acid ester or that are not in a drinkable or edible dosage form disclosed herein. These exemplary “low-dose” treatments may enable a lower volume needed to be swallowed or ingested though achieve the same or better pharmacokinetics in comparison to previously available ademetionine products administered on a bi-daily or greater schedule. Some embodiments relate to administration of a selected improved dosage on a once-a-day basis. In some embodiments, the once-a-day dose may be administered in a single dosage unit exemplified by a single composition of the invention. In other exemplary embodiments, the single dose may be administered as multiple composition of the invention taken at one time. In some embodiments, for instance, a dosage of about 400 to 3600 mg of ademetionine per day may be divided into two, three, four or more compositions of about 50 to 2000 mg, preferably about 100 to 1600 mg of ademetionine per unit. In some preferred embodiments, the daily dose may comprise two, three or four units (e.g. drinks) of about 100 to 800 mg of ademetionine per unit.

Exemplary drinkable or edible ademetionine compositions comprising a gallic acid ester may be configured to enable high bioavailability of the ademetionine. “High bioavailability” compositions are those which provide higher average maximum ademetionine blood plasma concentration (Cmax) and/or average ademetionine plasma area under the curve (AUC) values in comparison to the same dosage forms of ademetionine without the gallic acid ester or in comparison to other currently available commercial ademetionine formulations. High bioavailability compositions when dosed to a selected subject group provide an average Cmax of at least about 100 to 130 ng/mL (and/or an average AUC of at least about 500 ng·h/mL) per each 100 mg dosage of ademetionine ion. Thus in some embodiments, ademetionine drinkable or edible compositions comprising one or more gallic acid ester are provided in high bioavailability drinkable or edible ademetionine compositions.

In some exemplary embodiments, administration of drinkable or edible compositions disclosed herein to a selected subject group provides in said selected subject group an average ademetionine Cmax (average maximum plasma concentration) of at least about 100 ng/mL per each 100 mg of ademetionine ion, at least about 110 ng/mL per each 100 mg of ademetionine ion, or or at least about 120 ng/mL per each 100 mg of ademetionine ion, or of at least about 130 ng/mL per each 100 mg of ademetionine ion, or of at least about 150 ng/mL per each 100 mg of ademetionine ion, or of at least about 175 ng/mL per each 100 mg of ademetionine ion, or of at least about 200 ng/mL per each 100 mg of ademetionine ion, or of at least about 225 ng/mL per each 100 mg of ademetionine ion or of at least about 250 ng/mL per each 100 mg of ademetionine ion, or of at least about 300 ng/mL per each 100 mg of ademetionine ion. In some embodiments, administration of drinkable or edible compositions disclosed herein to a selected subject group provides in said selected subject group an average ademetionine Cmax of at least about 12 ng/mL, at least about 13 ng/mL, at least about 15 ng/mL, at least about 17.5 ng/mL, at least about 20 ng/mL, at least about 22.5 ng/mL, at least about 25 ng/mL, or at least about 30 ng/mL per each 10 mg of ademetionine ion. In other embodiments, administration of drinkable or edible compositions disclosed herein to a selected subject group provides in said selected subject group an average ademetionine Cmax of at least about 1.2 ng/mL, at least about 1.3 ng/mL, at least about 1.35 ng/mL, at least about 1.5 ng/mL, at least about 1.75 ng/mL, at least about 2.0 ng/mL, at least about 2.25 ng/mL, at least about 2.5 ng/mL, or at least about 3.0 ng/mL per each 1 mg of ademetionine ion. In certain specific embodiments, the gallic acid ester is selected from ethyl gallate, isoamyl gallate, propyl gallate and octyl gallate. Thus provided herein are drinkable or edible compositions comprising ademetionine and ethyl gallate, isoamyl gallate, propyl gallate and/or octyl gallate, wherein said compositions when administered to a selected subject group provides in said selected subject group an average ademetionine Cmax of at least about 1.2 ng/mL, at least about 1.3 ng/mL, at least about 1.35 ng/mL, at least about 1.5 ng/mL, at least about 1.75 ng/mL, at least about 2.0 ng/mL, at least about 2.25 ng/mL, at least about 2.5 ng/mL, or at least about 3.0 ng/mL per each 1 mg of ademetionine ion.

In some embodiments, administration of drinkable or edible compositions disclosed herein to a selected subject group provides in said selected subject group an average AUC of at least about 800 ng·h/mL per each 100 mg dosage of ademetionine ion, or of at least about 850 ng·h/mL per each 100 mg dosage of ademetionine ion, or at least about 900 ng·h/mL per each 100 mg dosage of ademetionine ion, at least about 950 ng·h/mL per each 100 mg dosage of ademetionine ion, or at least about 1000 ng·h/mL per each 100 mg dosage of ademetionine ion. In some embodiments, the administration of drinkable or edible compositions disclosed herein to a selected subject group provides in said selected subject group an average ademetionine AUC of at least about 80 ng·h/mL, at least about 85 ng·h/mL, at least about 90 ng·h/mL, at least about 95 ng·h/mL, or at least about 100 ng·h/mL per each 10 mg of ademetionine ion. In other embodiments, administration of drinkable or edible compositions disclosed herein to a selected subject group provides in said selected subject group an average ademetionine AUC of at least about 8 ng·h/mL, at least about 8.5 ng·h/mL, at least about 9 ng·h/mL, at least about 9.5 ng·h/mL, or at least about 10 ng·h/mL per each 1 mg of ademetionine ion. In a specific embodiment, the gallic acid ester is selected from ethyl gallate, isoamyl gallate, propyl gallate and octyl gallate. Thus provided herein are drinkable or edible compositions comprising ademetionine and ethyl gallate, isoamyl gallate, propyl gallate and/or octyl gallate, wherein said compositions when administered to a selected subject group provides in said selected subject group an average ademetionine AUC of at least about 8 ng·h/mL, at least about 8.5 ng·h/mL, at least about 9 ng·h/mL, at least about 9.5 ng·h/mL, or at least about 10 ng·h/mL per each 1 mg of ademetionine ion. In some embodiments, the dose of ademetionine ion delivered is at least 10 mg. In preferred embodiments, the dose of ademetionine ion delivered is from 10 to 3600 mg or 50 to 1600 mg.

In some embodiments, the term “selected subject group” is a group of selected human subjects. In some embodiments, a suitable “selected subject group” has six or more subjects who are dosed fasted. In some embodiments, all members of the “selected subject group” have pharmacokinetic parameters for ademetionine that fall within statistically normal ranges (i.e. no outliers) and no member will be included on the basis of non-standard or unusual ademetionine absorption or metabolism. In some embodiments, all members of the “selected subject group” are males. In other embodiments, the selected subject group is a group of selected non-human subjects. Preferably the non-human subjects are major food animals, companion animals or minor species animals. By “companion animals” it is meant to include animals such as, but not limited to, horses, dogs, and cats as recommended by the FDA. In some embodiments, administration of drinkable or edible compositions disclosed herein to a selected non-human subject group provides in said selected non-human subject group an average ademetionine Cmax of at least about 1000 ng/mL, at least about 1500 ng/mL, at least about 2000 ng/mL, at least about 2500 ng/mL, at least about 3000 ng/mL, or at least about 3500 ng/mL per each 100 mg of ademetionine ion. Thus provided herein are drinkable or edible compositions comprising ademetionine and ethyl gallate, isoamyl gallate, propyl gallate and/or octyl gallate, wherein administration of said compositions to a selected non-human subject group provides in said selected non-human subject group an average ademetionine Cmax of at least about 1000 ng/mL, at least about 1500 ng/mL, at least about 2000 ng/mL, at least about 2500 ng/mL, at least about 3000 ng/mL, or at least about 3500 ng/mL per each 100 mg of ademetionine ion.

In some embodiments, ademetionine and the at least one gallic acid ester are administered at the same time. In certain specific embodiments, ademetionine and the at least one gallic acid ester are co-formulated. In some exemplary embodiments, drinkable or edible compositions disclosed herein comprise ademetionine and at least one gallic acid ester, wherein said ademetionine and said at least one gallic acid ester are present in the initial mixture of the composition. In other embodiments, drinkable or edible compositions disclosed herein comprise ademetionine and at least one gallic acid ester, wherein said at least one gallic acid ester is present in separate compositions, for example separate milkshakes, suspensions, crystals, sprinkles or powders.

Also provided herein is a method for improving the pharmacokinetic parameters of ademetionine administered to a subject, said method comprising administering to the subject a drinkable or edible composition comprising at least one physiologically effective dosage of ademetionine in combination with at least one gallic acid ester selected to improve the pharmacokinetic parameters of said ademetionine in a subject. In some embodiments, said pharmacokinetic parameters are measurable in the subject by one of a Cmax, an AUC, and combinations thereof in comparison to a control group administered the same or similar ademetionine formulation yet lacking the gallic acid ester or being administered in drinkable or edible forms disclosed herein. For greater clarity all references to dose within this patent refer to dose as the dose of ademetionine ion. Pharmacokinetic parameters such as average maximum plasma concentration of ademetionine (Cmax) are determined using a bioanalytical method with adequate sensitivity, specificity, ruggedness, stability and repeatability (for example, a qualified liquid chromatography triple quad mass spectrometry based method coupled with a suitable extraction method for the separation of analyte from plasma). AUC values are preferably calculated from 0-24 hours using the trapezoid method and are uncorrected for baseline, endogenous ademetionine levels. A suitable “selected subject group” or “selected non-human subject group” has six or more subjects. In some embodiments said “selected subject group” or “selected non-human subject group” are dosed fasted. In some embodiments, all subjects are male subjects. All members of the “selected subject group” or “selected non-human subject group” have pharmacokinetic parameters for ademetionine that fall within statistically normal ranges (i.e. no outliers) and no member will be included on the basis of non-standard or unusual ademetionine absorption or metabolism which may or may not result from a different genetic profile. The average Cmax values are derived by averaging the concentration at each time point for all members of the subject group. Use of methods in vivo provides superior Cmax and/or AUC values in comparison to conventional dosage forms of ademetionine that are not drinkable or edible forms disclosed herein.

Some embodiments also relate to compositions and methods which yield a lower effective dose and/or less variable pharmacokinetic parameters (such as Tmax values with reduced variation) in comparison to conventional ademetionine compositions or other ademetionine compositions that lack gallic acid ester (“ademetionine control”). A “lower effective dose” or “reduced effective dose” is meant to define a physiologically acceptable dose of ademetionine which results in pharmacokinetic parameters which are equivalent (or better) to a significantly higher dose of another ademetionine composition, such as that obtained through administration of a higher dose of one or more commercially available or “control” ademetionine formulations. A “conventional” or “control” ademetionine formulation/composition are typically formulations/compositions which have the same or similar dose of ademetionine but are not drinkable or edible forms as disclosed herein or, in certain cases, are drinkable or edible forms as disclosed herein yet do not contain a gallic acid ester. Compositions such as those provided herein which exhibit similar Cmax and AUC values at lower ademetionine doses would have many benefits including a lower drinkable or edible volume, increased rate of onset and/or potentially increased tolerability and/or compliance.

Additional embodiments also relate to compositions and methods which yield an improved side effect profile in comparison to conventional non-drinkable or edible ademetionine formulations. An “improved side effect” or “reduced side effect” or “beneficial side effect” profile is meant to define improved tolerability to administration of ademetionine, such as less frequency and/or reduced intensity of side effects associated with ademetionine supplementation. It is further recognized by the present investigators that any observed negative side effects associated with ademetionine ion supplementation may be attributed to the ademetionine counterion(s) present in the ademetionine salts. By reducing the daily dose of ademetionine ion needed to experience a positive therapeutic outcome, the corresponding significant reduction in ademetionine counterion(s) may contribute to the improved side effect profile.

Some exemplary embodiments also relate to a dosing regimen of ademetionine of once daily, or QD dosing, which results in improved pharmacokinetic profiles of ademetionine in comparison to conventional twice daily or more frequent dosing. In certain embodiments, the effect of once a day dosing is believed to result in the most consistent pharmacokinetic parameter measurements, specifically those of the Cmax and Tmax. The less variable pharmacokinetic profiles that result from once a day dosing of formulations provided herein allow for more certainty of dosing and exposure by the medical practitioner as well as improved side effect profiles for subjects. Side effects include for example, nausea or stomach irritation, gastrointestinal upset, insomnia, headaches, irritation or possibly heart palpitations.

In some embodiments, drinkable or edible compositions which exhibit superior pharmacokinetic profiles in comparison to conventional ademetionine dosage forms provide an improved rate of onset of ademetionine which may result in enhanced therapeutic outcomes. Improved rate of onset is meant to mean the rate at which the subject experiences a positive outcome. For example, in Depression, the onset of antidepressant action is typically 4-6 weeks. Ademetionine formulations with improved pharmacokinetic profiles may be associated with corresponding improvement in therapeutic affect (e.g. antidepressant effect) in less than the typical or expected 4-6 weeks.

Other exemplary embodiments relate to methods for treating a disease or disorder in a subject and/or improving the nutritional status in a subject, said methods comprising administering to said subject drinkable or edible compositions comprising physiologically effective dosages of ademetionine. In some embodiments, said method further comprises administering to said subject drinkable or edible compositions comprising physiologically effective dosages of ademetionine in combination with one or more gallic acid ester thereby improving the pharmacokinetic profile of ademetionine. Improved pharmacokinetic profiles are identified by, for example, an increase in Cmax and/or AUC values; or alternatively a decrease in effective dose; or pharmacokinetic parameters with reduced variation. Achieving one or more of these criteria would constitute an improvement in the pharmacokinetic profile of ademetionine. In a specific embodiment, the gallic acid ester is selected from ethyl gallate, isoamyl gallate, propyl gallate and octyl gallate.

In some embodiments, there is provided a method of treating or preventing a disease condition or disorder, comprising administering to a subject in need of such treatment an effective amount of a drinkable or edible composition as described herein. In some embodiments, there is provided a method of treating in a patient a disease or disorder selected from the group consisting of mental and psychiatric disorders, nervous system diseases and disorders, neurological diseases and disorders, conditions associated with injuries to the central nervous system, liver diseases and disorders, cancers, joint diseases and disorders, inflammatory diseases and disorders, autoimmune diseases and disorders, degenerative diseases and disorders, soft-tissue diseases and disorders, pain diseases and disorders, cardiovascular disorders related to hyper-homocysteinemia and hypo-homocysteinemia, genetic disorders related to hyper-methylation and hypo-methylation, gastrointestinal diseases and disorders, atherosclerosis, Lesch-Nyhan disease, and disorders induced in whole or in part by oxidative or free-radical damage, comprising administering to the patient in need thereof a drinkable or edible composition as described herein. In some embodiments, the subject is human.

### Excipients and Processing Parameters

Various excipients may be suitable for use in generating drinkable or edible compositions disclosed herein. Also, the ademetionine drinkable and edible compositions disclosed herein may be processed or manufactured under specific conditions such as, for example, mixing methods (including sieve size and rpm), homogenization time, pasteurization conditions, environmental parameters (e.g. temperature and humidity) and combinations thereof.

Binders or Viscosity Control Agents

The binding material which holds the bulk of the product together is known as a “binder” or “granulator”. Binders suitable for use in the present invention are exemplified by, but are not limited to, sugars, gelatin, gums, microcrystalline cellulose and modified celluloses, waxes or synthetic polymers like polyethylene glycol or polyvinyl pyrrolidone. Some of these agents also act as viscosity control agents, in particular modified celluloses or carrageenans. Some of these agents also act as bulking agents in manufacturing gummy/gummie products; these typically are sucrose, maltose, fructose or corn syrup.

Lubricants

Lubricants are substances which aid in the manufacturing process as they help minimize clumping of the products and also help release them from the manufacturing machinery. Suitable lubricants for drinkable and edible composition disclosed herein include but are not limited to magnesium stearate, talc, calcium stearate, stearic acid (stearin), hydrogenated vegetable oils, sodium benzoate, leucine, carbowax 4000 and sodium stearyl fumarate. Further exemplary embodiments also relate to improved pharmacokinetic compositions comprising ademetionine and one or more gallic acid esters and one or more lubricants.

Glidants

Glidants, also referred to as “flow-aids”, help to keep the powder making up the products flowing as the products are being made, stopping them from forming lumps. Suitable glidants for drinkable and edible compositions disclosed herein include but are not limited to colloidal silicon dioxide, talc, calcium silicate and magnesium silicate. Additional embodiments relate to improved pharmacokinetic compositions comprising ademetionine and one or more gallic acid esters and one or more glidants.

Processing Methods and Parameters

Processing methods and/or parameters which may be modified in order to improve the pharmacokinetic profile and/or alter the dissolution profile of drinkable or edible ademetionine compositions include but are not limited to: relative humidity, temperature, homogenization and/or pasteurization time, and other environmental parameters.

Combinations with Ademetionine

Some exemplary embodiments of the present invention relate to combinations of ademetionine with one or more active ingredients that are commonly prescribed or used for treating and/or prophylaxis in a subject a disease or disorder selected from the group consisting of, but not limited to, a mental or psychiatric disorder (e.g. psychotic or non-psychotic mental disorders such as depression and substance abuse disorders, respectively), a nervous system disease/disorder (e.g. a central nervous system disease such as Alzheimer's, Progressive Supranuclear Palsy, or other Tauopathy disorders), other neurological disease/disorders (e.g. headaches and sleep disorders), conditions associated with injury to the central nervous system, a liver disease/disorder (e.g. alcoholic liver disease), a cancer (e.g. solid and blood-borne cancers, including those of the gastrointestinal tract), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper or hypo methylation, a gastrointestinal disease/disorder, a cardiovascular disease/disorder, and a disorder induced in whole or in part by oxidative or free-radical damage, comprising administering to said subject an exemplary drinkable and/or edible composition of the present invention. In some exemplary embodiments, the disease or disorder is depression. In specific embodiments, the depression is selected from major depressive disorder, major depression, clinical depression, anxiety depression, atypical depression, melancholic depression, catatonic depression, situational depression, reactive depression, late-life depression, Seasonal Affective Disorder (SAD), minor depression, postpartum depression, inflammatory depression, late-life depression, brief recurrent depression, mild depression, treatment-resistant depression (TRD), co-morbid depression, Parkinson's depression, and HIV-associated depression. In more specific embodiments, the depression is major depressive disorder. In other specific embodiments, the depression is treatment-resistant depression.

In some exemplary embodiments of the present invention relate to combinations of ademetionine with one or more active ingredients that are commonly prescribed or used for treatment of and/or prophylaxis of mental or psychiatric disorders in a subject include, but are not limited to, tricyclic antidepressants (TCAs), tetracyclic antidepressants, aminoketones, phenylpiperazines, selective serotonin reuptake inhibitors (SSRIs), monoamine oxidase inhibitors (MAOIs), serotonin-norepinephrine reuptake inhibitors (SNRIs), norepinephrine-serotonin reuptake inhibitors (NSRIs), dopamine reuptake inhibitors, norepinephrine-dopamine reuptake inhibitors, norepinephrine reuptake inhibitors, selective serotonin reuptake enhancers, noradrenergic and serotonin specific antidepressants, folate, folate derivatives (e.g. 1-methylfolate), omega fatty acids (e.g. omega-3 fatty acids), substance P receptor antagonists, neurokinin receptor antagonists such as saredutant, corticotrophin release factor antagonists such as mifepristone, atypical antipsychotics such as aripiprazole, commonly used antidepressant augmenters such as lithium or triple reuptake inhibitors.

Some exemplary embodiments of the present invention relate to combinations of ademetionine with one or more device therapies that are commonly prescribed or used for treatment of and/or prophylaxis of mental or psychiatric disorders in a subject include, but not limited to ECT (electro convulsive therapy) and electric shock therapy.

In some exemplary embodiments of the present invention relate to combinations of ademetionine with one or more active ingredients that are commonly prescribed or used for treatment of and/or prophylaxis of a nervous system disease/disorder in a subject include, but are not limited to anticonvulsants such as pregabalin, alpha-amino-3-hydroxy-5-methyl-4-isoxazolepropionic acid (AMPA) receptor antagonists, methylphosphonate (NMPA) receptor antagonists, histamine receptor antagonists, nitric oxide (NO) modulators, glutamate receptor antagonists, acetylcholinesterase inhibitors, dopamine agonists, N-methyl-d-aspartate (NMDA) receptor antagonists such as memantine, cholinesterase inhibitors such as donepezil, neuroprotectants, nootropic agents, CNS modulators, antiamyloidogenics.

In some exemplary embodiments of the present invention relate to combinations of ademetionine with one or more active ingredients that are commonly prescribed or used for treatment of and/or prophylaxis of a liver disorder in a subject include, but are not limited to, antiviral medication such as alpha interferon, ribavirin, lamivudine, steroids, antibiotics and zinc acetate.

In some exemplary embodiments of the present invention relate to combinations of ademetionine with one or more active ingredients that are commonly prescribed or used for treatment of and/or prophylaxis of a cancer in a subject include, but are not limited to, chemotherapeutic agents, drug resistance modulators, monoclonal antibodies, cytokines (e.g. interferons and interleukins), immunocytokines, growth factors, chemoprotectants, vaccines and other biological response modifiers.

In some exemplary embodiments of the present invention relate to combinations of ademetionine with one or more active ingredients that are commonly prescribed or used for treatment of and/or prophylaxis of a joint or inflammatory disease/disorder in a subject include, but are not limited to, analgesics, non-steroidal anti-inflammatory drug compounds (NSAID), disease-modifying antirheumatic drugs (DMARDs), corticosteroids, anakinra (an interleukin-1 receptor antagonist), COX-2 inhibition, gamma-aminobutyric acid-B (GABAB) receptor agonists, such as baclofen, GABAA potentiating drugs, such as the benzodiazepines tumor necrosis factor (TNF)-inhibiting drugs, and other drugs that modify the immune response (immunosuppressive drugs).

In some exemplary embodiments of the present invention relate to combinations of ademetionine with one or more active ingredients that are commonly prescribed or used for treatment of and/or prophylaxis of an autoimmune disease/disorder in a subject include, but are not limited to, DMARDs, corticosteroids, anakinra (an interleukin-1 receptor antagonist), TNF-inhibiting drugs, and other drugs that modify the immune response (immunosuppressive drugs).

In some exemplary embodiments of the present invention relate to combinations of ademetionine with one or more active ingredients that are commonly prescribed or used for treatment of and/or prophylaxis of a degenerative disease/disorder in a subject include, but are not limited to, NSAIDs, COX-2 inhibition, GABAB receptor agonists, such as baclofen, and GABAA potentiating drugs, such as the benzodiazepines.

In some exemplary embodiments of the present invention relate to combinations of ademetionine with one or more active ingredients that are commonly prescribed or used for treatment of and/or prophylaxis of a soft tissue disease/disorder in a subject include, but are not limited to, milnacipram, pregabalin, SNRIs, NSRIs, muscle relaxers, sedatives, painkillers, and NSAIDs.

In some exemplary embodiments of the present invention relate to combinations of ademetionine with one or more active ingredients that are commonly prescribed or used for treatment of and/or prophylaxis of a genetic disease/disorder related to hyper or hypo methylation in a subject include, but are not limited to methionine, MTA (5′-deoxy-5′-(methylthio) adenosine) and other ademetionine metabolites.

In some exemplary embodiments of the present invention relate to combinations of ademetionine with one or more active ingredients that are commonly prescribed or used for treatment of and/or prophylaxis of a gastrointestinal disease/disorder in a subject include, but are not limited to, 5-Aminosalicylic acid (5-ASA) medications, Corticosteroids (prednisone), immunomodulatory medications such as Azathioprine (Immuran), 6-Mercaptopurine (6-MP), Methotrexate and Cyclosporine (Sandimmune), commonly used antibiotics such as Metronidazole (Flagyl) and Ciprofloxacin (Cipro) and biologic agents such as Infliximab (Remicade).

In some exemplary embodiments of the present invention relate to combinations of ademetionine with one or more active ingredients that are commonly prescribed or used for treatment of and/or prophylaxis of a cardiovascular disease/disorder in a subject include, but are not limited to, statins, angiotensin-converting enzyme (ACE) inhibitors, ASA, ademetionine break down products such as methionine, MTA and folate, cardioprotectants, vasoprotectants, coagulation inhibitors.

In some exemplary embodiments of the present invention relate to combinations of ademetionine with one or more active ingredients that are commonly prescribed or used for the treatment of and/or prophylaxis of a metabolic disease in a subject including, but not limited to sulfonylureas, biguanides, thiazolidinediones, alpha-glucosidase inhibitors, meglitinides, incretin-based therapies, and DPP-4 inhibitors.

In some exemplary embodiments of the present invention relate to combinations of ademetionine with one or more active ingredients that are commonly prescribed or used for treatment of and/or prophylaxis of a disorder induced in whole or in part by oxidative or free-radical damage including, but are not limited to, antioxidants such as Vitamin A, Vitamin C, Vitamin E, polyphenols, flavonoids, selenium, carotenoids.

In some exemplary embodiments of the present invention relate to combinations of ademetionine with one or more active ingredients that are commonly prescribed or used for treatment of and/or prophylaxis of a disorder induced in whole or in part by damage to the central nervous system such as brain injury or spinal cord injury including, but not limited to, neuroprotectants, nootropic agents, CNS modulators, analgesics, muscle relaxants, apoptosis inhibitors, bone modulators, antioxidants.

In some exemplary embodiments of the present invention relate to combinations of ademetionine with methionine, MTA, folate, folate derivatives (e.g. 1-methylfolate), an omega fatty acid (e.g. Omega-3), vitamin B6 and/or B12. These agents may be correlated with lowering homocysteine production. Therefore, it is considered that combining ademetionine with methionine, MTA, folate, vitamin B6 and/or B12 may result in increased supplementation of ademetionine by enhancing the body's natural ability to make ademetionine while at the same time supplementing ademetionine with ademetionine exhibiting enhanced absorption and improved bioavailability. As used herein the term “folate” refers to vitamin B9 in all of its natural and synthetic forms including, but not limited to, folic acid, tetrahydrofolate and L-methylfolate.

In some embodiments, an exemplary ademetionine dosage form according to the invention may be included in a kit with a separate dosage form containing at least one other active ingredient, exemplified by one or more compounds suitable for the treatment of or commonly prescribed or used for the treating and/or prophylaxis in a subject a disease or disorder selected from the group consisting of, but not limited to, a mental or psychiatric disorder (e.g. psychotic/mood or non-psychotic mental disorders such as depression and substance related disorders, respectively), a nervous system disease/disorder (e.g. a central nervous system disease such as Alzheimer's), other neurological disease/disorders (e.g. headaches and sleep disorders), conditions associated with injury to the central nervous system, a liver disease/disorder (e.g. alcoholic liver disease), a cancer (e.g. solid and blood-borne cancers, including cancers of the gastrointestinal tract), a joint disease/disorder (e.g. arthritis), an inflammatory disease/disorder (e.g. ulcerative colitis), an autoimmune disease/disorder (e.g. systemic lupus erythematosis and rheumatoid arthritis), a degenerative disease/disorder (e.g. Amyotrophic Lateral Sclerosis), a soft-tissue disease/disorder (e.g. a fibromyalgia disorder), a pain disease/disorder, a genetic disorder related to hyper or hypo methylation, a gastrointestinal disease/disorder, a cardiovascular disease/disorder, and a disorder induced in whole or in part by oxidative or free-radical damage, comprising administering to said subject an exemplary composition of the present invention.

In addition to combinations of ademetionine with the one or more additional ingredients exemplified above or omega fatty acids, methionine, MTA, folate, 1-methylfolate, Omega 3, vitamin B6 and/or B12, administration of the exemplary ademetionine formulations of the invention may also augment the effects of other drugs or nutritional supplements being taken by the subject. Thus, some exemplary embodiments of the present invention relate to combinations of ademetionine formulations with drugs or nutritional compounds already employed for treating other diseases for increasing the activity of said drugs or nutritional compounds.

### PREFERRED EMBODIMENTS WITH ADEMETIONINE

Some exemplary embodiments of the invention include, but are not limited to, the following:

In embodiment 1, provided is a drinkable and/or edible composition comprising ademetionine. In embodiment 2, provided is a drinkable and/or edible composition comprising ademetionine of embodiment 1 wherein said composition comprises a milkshake, smoothie, slushie, bubble tea, pop, soda, crystal, powder, bubble tea, sprinkle, slurry, suspension, or other instant particulate oral dosage form. In embodiment 3, provided is a drinkable and/or edible composition comprising ademetionine of embodiment 1 wherein said composition comprises a snack bar, protein bar, meal-replacement bars, supplement bar, wafer, cracker, cookie, soup, cereal, cereal bar, cereal cluster, cake, yogurt, tapioca ball, pearl, baked good, gummy, chewy product, confectionary product, sprinkle or granola. In embodiment 4, provided is a drinkable and/or edible composition comprising ademetionine of embodiment 1, 2 and/or 3 which comprises ademetionine particles wherein at least about 50%, 60%, 70%, 80%, 90% or greater than 90% of said particles are from about 10 to about 5000 microns. In embodiment 5, provided is a drinkable and/or edible composition comprising ademetionine of embodiment 1, 2 and/or 3 which comprises ademetionine particles wherein at least about 50%, 60%, 70%, 80% or 90% of said particles are from about 10 to 50 microns, 50 to 100 microns, 100 to 200 microns, 200 to 300 microns, 300 to 400 microns, 400 to 500 microns, 500 to 600 microns, 600 to 700 microns, 700 to 800 microns, 800 to 900 microns or about 900 to 1000 microns. In embodiment 6, provided is a drinkable and/or edible composition comprising ademetionine of embodiment 4 and/or 5 which comprises wherein said particle size is measured by light scattering or dynamic light scattering. In embodiment 7, provided is a drinkable and/or edible composition comprising ademetionine of any of the embodiments 1-6 which further comprises one or more gallic acid esters. In embodiment 8, provided is a drinkable and/or edible composition comprising ademetionine of embodiment 7, wherein said one or more gallic acid ester is selected from methyl gallate, ethyl gallate, propyl gallate, butyl gallate, isobutyl gallate, isoamyl gallate, octyl gallate, dodecyl gallate, and hexadecyl gallate. In embodiment 9, provided is a drinkable and/or edible composition comprising ademetionine of embodiment 8, wherein said one or more gallic acid ester is selected from the ethyl gallate, propyl gallate, isoamyl gallate, and octyl gallate. In embodiment 10, provided is a drinkable and/or edible composition comprising ademetionine of embodiment 7, wherein the ratio (weight:weight) of gallic acid ester to ademetionine is from 5:1 to 1:400 or 1:1 to 1:100. In embodiment 11, provided is a drinkable and/or edible composition comprising ademetionine of embodiment 7, wherein said composition comprises from about 1 to 400 mg of said gallic acid ester. In embodiment 12, provided is a drinkable and/or edible composition comprising ademetionine of embodiment 7, wherein said composition comprises 0.1 to 80%, 0.25 to 50%, 0.25 to 25%, 0.25 to 10%, 0.5 to 10%, 10-20%, 20-30%, 30-40% or greater than 40% by weight gallic acid ester. In embodiment 13, provided is a process for producing a shelf-ready ademetionine milkshake composition comprising:
(a) preparing a milkshake mixture comprising a milk component and a starch component, wherein said starch component is a modified food starch;
(b) pasteurizing the mixture to form a pasteurized mixture;
(c) homogenizing the mixture at a pressure of from about 2,000 to about 5,000 psi to form a homogenized, pasteurized mixture;
(d) mixing the homogenized, pasteurized mixture with stabilized ademetionine and
(e) dispensing the combined mixture into separate, sterilized containers.
   In embodiment 14, provided is a process for producing a shelf-ready ademetionine drink crystal or powder composition comprising:
(a) preparing a mixture comprising an antifoaming agent and a flavorant;
(b) combining and then continuously stirring the above mixture with sugar;
(c) drying the resulting slurry;
(d) grinding the obtained dried crystals to a desired size crystal or powder;
(e) mixing the ground crystals or powder with stabilized ademetionine; and
(f) dispensing the ademetionine drink crystals into separate, sterilized packages.
   In embodiment 15, provided is a process for producing an ademetionine gummy composition comprising:
(a) preparing a mixture comprising unflavored gelatin, flavored gelatin, and cold water;
(b) stir mixture in heating pan until well mixed;
(c) continue stirring over medium heat until gelatin dissolves;
(d) remove from heat and let cool;
(e) add stabilized ademetionine and pour into desired casts or molds and leave to harden; and/or,
(f) optionally place at below 0° C. to solidify.
   In embodiment 16, provided is a process for producing an ademetionine granola bar composition comprising:
(a) preparing a granola base product comprising a dry ingredient mixture of 35% by weight rolled oats, 35% by weight rice crisp, 5% by weight almonds and 25% by weight peanuts; the ingredients are mixed and then granulated and then baked at about standard room temperatures and then granulated;
(b) preparing a binder comprising a mixture of approximately 65% by weight high maltose corn syrup, approximately 19% by weight honey, approximately 7% fructose, approximately 5% by weight canola oil, approximately 3% by weight maltodextrin, approximately 0.65% by weight salt, and approximately 0.35% by weight flavored powder; the binder is formed by mixing high maltose corn syrup and honey at standard room temperature until well mixed and then adding the additional components of the binder and mixing at room temperature until well-mixed;
(c) mix about 3% by weight stabilized ademetionine to about 57% by weight binder and about 40% by weight of the granola base product
(d) using a cold form sheeting process at a temperature of about 140-160° F. to press out the granola bars; and
(e) mechanically cut the granola bar sheet to smaller bars weighing approximately 40 grams each and having dimensions of about 100×30×16 mm.
   In embodiment 17, provided is a process for producing an ademetionine yogurt composition comprising:
(a) dispensing a yogurt or yogurt-containing product into a first chamber of a multi-chamber container;
(b) adding stabilized ademetionine to a second chamber (optionally comprising a topping) of said multi-chamber container;
(c) sealing the top of each chamber such that the stabilized methionine and yogurt or yogurt-containing product cannot come in contact with each other until at least the seal is removed;
(d) packaging the multi-chamber ademetionine yogurt composition for individual consumption; and,
(e) optionally shipping the ademetionine yogurt composition prior to refrigerator storage.
   In embodiment 18, provided is a process for producing a shelf-ready ademetionine marble soda composition comprising:
(a) filling a ramune or similar codd-neck bottle with a carbonated liquid that is optionally flavored;
(b) above the marble, adding stabilized ademetionine which is protected in a soluble membrane such that it will not drop below the marble into the carbonated liquid until the marble is released;
(c) adding the topper to the bottle;
(d) sealing the topper/bottle combination; and,
(e) optionally shipping the ademetionine marble soda composition prior to storage.
   In embodiment 19, provided is a process for producing an ademetionine bubble tea composition comprising:
(a) preparing a tea for consumption as a bubble tea which optionally comprises a milk component;
(b) preparing tapioca balls/pearls comprising stabilized ademetionine;
(c) adding said tapioca balls to said tea prior to drinking; and,
(d) dispensing said tea and ademetionine-containing balls into a container suitable for drinking from.
   In embodiment 20, provided is a process for preparing ademetionine in a multi-compartment container comprising:
(a) using a parfait cup described in US patent application number 2011/0303678;
(b) dispensing stabilized ademetionine into one chamber of said cup;
(c) dispensing a second edible or drinkable product into a second chamber of said cup; and,
(d) sealing the top of each chamber such that the contents of each chamber of physically separated.
   In embodiment 21, provided is a composition comprising ademetionine that is made by a process of any of embodiments 13 to 20. In embodiment 22, provided is a composition of any of embodiments 1 to 12 or 21, wherein the composition when administered to a selected subject group provides in said selected subject group an average maximum ademetionine blood plasma concentration (average Cₘₐₓ) of at least about 1.2 ng/mL per each 1 mg dosage of ademetionine ion. In embodiment 23, provided is a composition of any of embodiments 1 to 12 or 21, wherein the composition when administered to a selected subject group provides in said selected subject group an average ademetionine Cₘₐₓ of at least about 1.3 ng/mL per each 1 mg dosage of ademetionine ion. In embodiment 24, provided is a composition of any of embodiments 1 to 12 or 21, wherein the composition when administered to a selected subject group provides in said selected subject group an average ademetionine plasma area under the curve (average AUC) of at least about 8 ng·h/mL per each 1 mg dosage of ademetionine ion. In embodiment 25, provided is a composition of any of embodiments 1 to 12 or 21, wherein the composition comprises ademetionine and a gallic acid ester and wherein said composition when administered to a selected subject group provides in said selected subject group an average ademetionine AUC of at least about 8.5 ng·h/mL per each 1 mg of ademetionine ion. In embodiment 26, provided is a composition of any of embodiments 1 to 12 or 21, wherein the composition when administered to a selected subject group provides in said selected subject group one of an average Tₘₐₓ or Cₘₐₓ with reduced variation in comparison to a control group. In embodiment 27, provided is a composition of any of embodiments 1 to 12 or 21, wherein the composition when administered to a selected subject group through once a day dosing provides in said selected subject group an improved ademetionine pharmacokinetic profile in comparison to bi-daily or more frequent dosing of conventional ademetionine formulations in a control group. In embodiment 28, provided is a composition of any of embodiments 1 to 12 or 21, wherein the composition when administered to a selected subject group provides in said selected subject group a reduced side effect profile in comparison to a control group. In embodiment 29, provided is a composition of any of embodiments 1 to 12 or 21, wherein the composition when administered to a selected subject group provides in said selected subject group an improved rate of onset of ademetionine supplementation in comparison to a control group. In embodiment 30, provided is a composition of any of embodiments 1 to 29, wherein the composition comprises at least 10 mg, at least 25 mg or at least 50 mg ademetionine. In embodiment 31, provided is a composition of any of embodiments 1 to 29, wherein said composition comprises at least 100 mg ademetionine. In embodiment 32, provided is a composition of any of embodiments 1 to 29, wherein said composition comprises at least 200 mg ademetionine. In embodiment 33, provided is a composition of any of embodiments 1 to 29, wherein said composition comprises at least 400 mg ademetionine. In embodiment 34, provided is a composition of any of embodiments 1 to 29, wherein said composition comprises at least 5%, at least 10%, at least 15%, at least 20%, at least 25%, at least 30%, at least 35%, at least 40%, at least 45%, at least 50%, or at least greater than 50% by weight ademetionine ion. In embodiment 35, provided is a composition of any of embodiments 1 to 29, wherein said composition comprises from 10-3600 mg ademetionine. In embodiment 36, provided is a composition of any of embodiments 1 to 29, wherein said composition comprises from 10-2000 mg, 10-1600 mg, 50-2000, 50-1600, 100-2000, or 100-1600 mg ademetionine. In embodiment 37, provided is a method of treating a disease or disorder selected from the group consisting of a mental or psychiatric disorder, nervous system disease or disorder, neurological disease or disorder, condition associated with injury to the central nervous system, liver disease or disorder, cancer, joint disease or disorder, inflammatory disease or disorder, autoimmune disease or disorder, degenerative disease or disorder, soft-tissue disease or disorder, pain disease or disorder, genetic disorder related to hyper- or hypo-methylation, gastrointestinal disease or disorder, cardiovascular disease or disorder, atherosclerosis, Lesch-Nyhan disease, and disorder induced in whole or in part by oxidative or free-radical damage comprising administering a composition of any of embodiments 1-36 to a subject in need thereof. In embodiment 38, provided is a method of increasing the rate of onset of treatment of a disease or disorder selected from the group consisting of a mental or psychiatric disorder, nervous system disease or disorder, neurological disease or disorder, condition associated with injury to the central nervous system, liver disease or disorder, cancer, joint disease or disorder, inflammatory disease or disorder, autoimmune disease or disorder, degenerative disease or disorder, soft-tissue disease or disorder, pain disease or disorder, genetic disorder related to hyper- or hypo-methylation, gastrointestinal disease or disorder, cardiovascular disease or disorder, atherosclerosis, Lesch-Nyhan disease, and disorder induced in whole or in part by oxidative or free-radical damage comprising administering a composition of any of embodiments 1-36 to a subject in need thereof. In embodiment 39, provided is the method of embodiment 37 or 38, wherein the mental or psychiatric disorder is selected from the group consisting of an anxiety disorder, schizophrenia, major depressive disorder, major depression, clinical depression, dysthymic disorder, anxiety depression, atypical depression, melancholic depression, catatonic depression, situational depression, reactive depression, late-life depression, Seasonal Affective Disorder (SAD), minor depression, postpartum depression, inflammatory depression, late-life depression, brief recurrent depression, mild depression, treatment-resistant depression (TRD), co-morbid depression, Parkinson's depression, HIV-associated depression, multi-infarct dementia, and bipolar disorder;
the inflammatory disease or disorder is selected from the group consisting of systemic lupus, inflammatory bowel disease, allergic rhinitis, contact dermatitis, asthma, autoimmune hepatitis, and pelvic inflammatory disease;
the cardiovascular disease or disorder is selected from the group consisting of hyper- or hypo-homocysteinemia, coronary heart disease, stroke, peripheral vascular disease, and atherosclerotic disease;
the depressive disorder is a co-morbid depression arising in a subject who is or has been undergoing treatment for one or more diseases or disorders selected from the group consisting of cancer, Parkinson's disease, and HIV;
the nervous system disease or disorder or injury is selected from the group consisting of Parkinson's disease, Alzheimer's disease, and cognitive impairment;
the liver disease or disorder is selected from the group consisting of alcoholic liver disease, non-alcoholic fatty liver disease, viral or non-viral hepatitis, liver cancer, oxidative liver disease, drug induced liver injury, cholestasis, and cirrhosis;
the cancer is selected from the group consisting of liver cancer, bowel cancer, colon cancer, rectal cancer, colorectal, stomach cancer, esophageal cancer, and adenocarcinoma;
the joint disease or disorder is arthritis or osteoarthritis;
the soft-tissue disease or disorder is fibromyalgia;
the pain disease or disorder is fibromyalgia or abdominal pain; or the genetic disorder related to hyper- or hypo-methylation is methylenetetrahydrofolate reductase deficiency.

In embodiment 40, provided is the method of embodiment 37 or 38, wherein the subject has fasted prior to administration of said composition. In embodiment 41, provided is a method of making a composition for improved appealability of ademetionine, wherein said method comprises formulating ademetionine into a drinkable or edible dosage form. In embodiment 42, provided is a method for improving the appealability of ademetionine, wherein said method comprises administering to said subject an exemplary drinkable or edible composition which provides a physiologically effective amount of ademetionine. In embodiment 43, provided is a method for ingesting ademetionine, wherein said method comprises administering to a subject a drinkable or edible composition which provides a physiologically effective amount of ademetionine. In embodiment 44, provided is a method of any one of embodiments 41-43, wherein said drinkable or edible composition comprises at least one gallic acid ester. In embodiment 45, provided is the method of embodiment 44, wherein said at least one gallic acid ester is selected from methyl gallate, ethyl gallate, propyl gallate, butyl gallate, isobutyl gallate, isoamyl gallate, octyl gallate, dodecyl gallate, and hexadecyl gallate. In embodiment 46, provided is the method of embodiment 44, wherein said at least one gallic acid ester is selected from ethyl gallate, isoamyl gallate, propyl gallate and octyl gallate. In embodiment 47, provided is the composition of any of embodiments 1-36, wherein said composition from 5 to 10%, 10 to 20%, 20 to 30%, 30 to 40%, 40 to 50%, 50 to 60%, 60 to 70%, 70 to 80%, 80 to 90%, 90 to 95% or 95 to 99% by weight ademetionine. In embodiment 48, provided is the composition of any of embodiments 1-36, wherein said composition is not a capsule. In embodiment 49, provided is the composition of any of embodiments 1-36, wherein said composition is not a tablet. In embodiment 50, provided is the composition of any of embodiments 1-36, wherein said composition is not a mini-tablet. In embodiment 51, provided is the composition of any of embodiments 1-36, wherein said composition does not comprise dissolved oxygen. In embodiment 52, provided is the composition of any of embodiments 1-36, wherein said composition does not comprise ten or more amino acids. In embodiment 53, provided is the composition of any of embodiments 1-36, wherein said composition does not comprise ten or more enzymes. In embodiment 54, provided is the composition of any of embodiments 1-36, wherein said composition does not comprise one or more aerobic proteins. In embodiment 55, provided is the composition of any of embodiments 1-36, wherein said composition does not comprise stabilized rice bran. In embodiment 56, provided is the composition of any of embodiments 1-36, wherein said composition does not comprise calcium propionate. In embodiment 57, provided is the composition of any of embodiments 1-36, wherein said composition does not comprise CellFood™. In embodiment 58, provided is the composition of any of embodiments 1-36, wherein said composition further comprises gelatin. In embodiment 59, provided is the composition of any of embodiments 1-36, wherein said composition further comprises chicle. In embodiment 60, provided is the composition of any of embodiments 1-36, wherein said composition further comprises gum arabic. In embodiment 61, provided is the composition of any of embodiments 1-36, wherein said composition further comprises a butadiene-based synthetic rubber. In embodiment 62, provided is the composition of any of embodiments 1-36, wherein said composition is a gummy or chewy product (including a carbohydrate and/or energy chew and/or gel), a confectionary product (including bakers' confections and sugar confections including candies, pastilles and sweets). In embodiment 63, provided is a drinkable and/or edible composition comprising ademetionine and gelatin. In embodiment 64, provided is a drinkable and/or edible composition comprising ademetionine, gelatin and one or more gallic acid esters. In embodiment 65, provided is a drinkable and/or edible composition comprising ademetionine and one or more milk or modified milk products. In embodiment 66, provided is a drinkable and/or edible composition comprising ademetionine, one or more milk or modified milk products and one or more gallic acid esters. In embodiment 67, provided is a drinkable and/or edible composition comprising ademetionine and dissolved carbon dioxide. In embodiment 68, provided is a drinkable and/or edible composition comprising ademetionine, dissolved carbon dioxide and one or more gallic acid esters. In embodiment 69, provided is the composition of embodiment 1, wherein said composition is not designed for dogs and/or cats. In embodiment 70, provided is the composition of any of embodiments 1-68, wherein said composition is recommended for human consumption. In embodiment 71, provided is the method of embodiment 37 or 38, wherein the mental or psychiatric disorder is selected from the group consisting of an anxiety disorder, schizophrenia, major depressive disorder, major depression, clinical depression, dysthymic disorder, anxiety depression, atypical depression, melancholic depression, catatonic depression, situational depression, reactive depression, late-life depression, Seasonal Affective Disorder (SAD), minor depression, postpartum depression, inflammatory depression, late-life depression, brief recurrent depression, mild depression, treatment-resistant depression (TRD), co-morbid depression, Parkinson's depression, HIV-associated depression, multi-infarct dementia, and bipolar disorder. In embodiment 72, provided is the method of embodiment 71, wherein the mental or psychiatric disorder is major depressive disorder. In embodiment 73, provided is the method of embodiment 71, wherein the mental or psychiatric disorder is treatment-resistant depression. In embodiment 74, provided is the composition of embodiment 1, wherein said composition is an instant coffee beverage. In embodiment 75, provided is the composition of embodiment 1, wherein said composition is an instant soup. In embodiment 76, provided is the composition of embodiment 1, wherein said composition does not comprise formulations for systemic buccal delivery. In embodiment 77, provided is the composition of embodiment 76, wherein said composition does not comprise formulations for systemic buccal delivery comprising as active principle sulpho-adenosyl-L-methionine, in particular in the form of chewing gums, chewable tablets, orodispersible tablets and oromucosal preparations capable of allowing the absorption of said active principle through the oral mucosa. In embodiment 78, provided is a drinkable and/or edible composition comprising ademetionine and dissolved nitrogen. In embodiment 79, provided is a drinkable and/or edible composition comprising ademetionine, dissolved nitrogen and one or more gallic acid esters. In embodiment 80, provided is a drinkable and/or edible composition comprising ademetionine, dissolved carbon dioxide and one or more gallic acid esters. Within other related aspects a delivery device is provided for providing a drinkable beverage. Within one embodiment the delivery device is a multi-chambered delivery device. Within one embodiment the delivery device comprises a first and a second chamber. Within further embodiments the first chamber is a dry chamber which contains an ademetionine composition as described in any one of embodiments 1 to 80. Within a preferred embodiment the first chamber having an ademetionine composition is under a vacuum, or contains a gas such as carbon dioxide, nitrous oxide or nitrogen. Within the second other chamber is a liquid (e.g., soda, milk, tea, water) or semi-liquid (e.g., yogurt, milkshake or soup). Prior to ingestion the first chamber and second chamber are admixed, and the mixed composition ingested.

The present embodiments are further described by the following examples. These examples, while illustrating certain specific aspects of the present embodiments, should not be considered to limit or circumscribe the scope of the disclosed embodiments.

### EXAMPLES

### Example 1

### Drinkable Ademetionine Compositions

A suspension for a shelf-ready ademetionine milkshake is prepared by mixing a milk component and a starch component. The mixture is then pasteurized and homogenized using conventional methods in the art, for example using the plate-in-frame HTST pasteurization process. The temperature for pasteurization is preferably from about 70° C. to about 82° C. Using lower temperatures may cause the starch granules to swell which is undesirable since these swollen granules often sheer off during the homogenization step thus altering the texture and consistency. Using higher temperatures may negatively affect the taste of the milkshake. Once pasteurized, the mixture is homogenized, preferably under a total pressure of from about 2,000 to about 5,000 psi. A total pressure of from about 3,000 to about 4,500 psi is more preferred. The use of lower pressures may result in undesirable instability, whereas the use of higher pressures may be impractical. Viscosity control agents such as carrageenan and sodium carboxymethylcellulose may be added prior to homogenization. Any conventional homogenization apparatus or technique can be employed in preparing the milkshake compositions of the present invention. In carrying out the homogenization step it is preferable to use temperatures ranging from about 38° C. to about 55° C., as lower temperatures may not adequately homogenize the milk component.

The homogenized mix is then cooled and mixed with a stabilized ademetionine composition (made according to GMP procedures and optionally includes a gallic acid ester) and dispensed into sterilized containers (e.g., cans or bottles) to a level of 90% of the total volume of each container. The headspace of the container is filled with air and the containers sealed. The sealed containers are, optionally, then sterilized at a temperature and pressure that will not accelerate ademetionine degradation. The viscosity of the final milkshake composition is then tested by conventional taste-tests.

In alternative methods, a frozen or refrigerated ademetionine milkshake is prepared by freezing at least two portions of its constituents under different conditions, such that clearly different ice crystal sizes are generated, and freezing the combined portions to a storing temperature below −15° C.; this frozen product is prepared for consumption by partly thawing it, using a controlled amount of heat or microwave energy.

High bioavailability ademetionine milkshakes are also prepared using ademetionine mixed with one or more the gallic acid ester which is selected from ethyl gallate, isoamyl gallate, propyl gallate and/or octyl gallate.

### Example 2

### Ademetionine Drink Crystals

Dry ademetionine drink crystals are prepared using methyl silicone antifoaming agent having 30% silicone in water as well as a flavorant containing a commercially available base (for example a cola base or fruit-flavored base) to make a homogeneous solution. The solution is then stirred with sugar. The resulting slurry is then spread on stainless steel pans and vacuum-dried under conditions designed for adequate drying. The dried crystalline material is then ground and sieved to obtain crystals of desired size. Some crystals are further ground into powder material.

The dried drink crystals or powders are then mixed with stabilized ademetionine and packaged into small, colorful sachets or packets which are convenient for on-the-go use and can be sold in either individual packets or large-scale boxes of various sizes with multiple, individual packets. Various dried (vacuum and/or freeze-dried) drink crystals or powders may be mixed with ademetionine including, for example, milk products, yogurt products, coffee products, dried or instant soup products, fruit products, vegetable products, vitamin products, mineral products, and/or combinations thereof. Other types of formulation methods are suitable for ademetionine to be packed into sachets or packets that contain pre-made drink crystals or to be mixed with pre-made drink crystals using methods such as those described in U.S. Pat. No. 6,270,804, which is incorporated herein by reference. Particularly useful packaging is described in U.S. Pat. No. 7,757,855, which is also incorporated herein by reference.

High bioavailability ademetionine drink crystals or powders are also prepared using one or more gallic acid esters mixed with ademetionine. In a specific embodiment, the gallic acid ester is selected from ethyl gallate, isoamyl gallate, propyl gallate and octyl gallate.

### Example 3

### Ademetionine Gummies

Gummy or chewy ademetionine products are prepared using water (approximately 15-20% by weight) which is heated to boiling and 10% oxidized starch (by weight) is added, along with 10% gum arabic (by weight). The mixture is placed in a high speed mixer to adequately dissolve the solid products. The oxidized starch gelatinizes on dispersion in the excess hot water and then a small amount of fat is added (approximately 5% by weight), along with an emulsifier, flavourant and desired colouring agent. The solution is brought to boiling (approximately 115° C.) until all components dissolve. The hot solution is cooled, stabilized ademetionine is added, and the complete solution is then poured into moulds or casts of desired shape (for example a small cylindrical or cube shape) and left to cool completely.

The cooled, uniformly shaped ademetionine gummies are packaged and stored at room temperature.

High bioavailability ademetionine gummies are also prepared using one or more gallic acid esters mixed with ademetionine. In a specific embodiment, the gallic acid ester is selected from ethyl gallate, isoamyl gallate, propyl gallate and octyl gallate.

### Example 4

### Ademetionine Granola Bars

Ademetionine granola products are prepared using 100-3600 mg of ademetionine.

A granola base product is first made using a dry ingredient mixture of 35% by weight rolled oats, 35% by weight rice crisp, 5% by weight almonds and 25% by weight peanuts. The dry ingredients are mixed and granulated. The mixture is baked at about standard room temperatures and then granulated.

A binder also used in the process is made using a mixture of approximately 65% by weight high maltose corn syrup, approximately 19% by weight honey, approximately 7% fructose, approximately 5% by weight canola oil, approximately 3% by weight maltodextrin, approximately 0.65% by weight salt, and approximately 0.35% by weight flavored powder. The binder is formed by mixing high maltose corn syrup and honey at standard room temperature until well mixed and then adding the additional components of the binder and mixing at room temperature until well-mixed.

About 3% by weight ademetionine (stabilized) is then mixed with the about 57% by weight binder and about 40% by weight of the granola base product and a chewy granola product containing the stabilized ademetionine is made using a cold form sheeting process at low temperatures so as to minimize ademetionine racemization and/or degradation. The chewy granola product containing the coated ademetionine is then mechanically cut into bars weighing approximately 40 grams and having dimensions of about 100×30×16 mm.

The uniformly shaped granola bars are packaged and/or shipped and may be stored at room temperature.

In another process of this invention, ademetionine is added to the granola bars above during a coating process rather than mixed prior to being pressed. In this aspect, the granola/binder base is formed using the cold sheeting process as above and then a coating comprising ademetionine is applied to the chewy granola product and then the chewy granola product is mechanically cut into bars as detailed above. Alternatively, the chewy granola product is mechanically cut into bars first and then the ademetionine coating composition is applied to the entirety of the granola product.

High bioavailability ademetionine granola bars are also prepared using one or more gallic acid esters mixed with ademetionine. In a specific embodiment, the gallic acid ester is selected from ethyl gallate, isoamyl gallate, propyl gallate and octyl gallate.

### Example 5

### Ademetionine Yogurt Parfaits

Preferred multi-compartment ademetionine yogurt products are prepared using 100-3600 mg of ademetionine. A yogurt or yogurt-containing product is dispensed into a first chamber of a 2-chamber parfait container; stabilized ademetionine is added into a second chamber (optionally comprising a topping) of said container; the top of each chamber is sealed such that the stabilized methionine and yogurt or yogurt-containing product cannot come in contact with each other until the seal is removed; the sealed multi-chamber ademetionine yogurt compositions are packaged; and, optionally shipped the prior to refrigerator and/or freezer storage.

High bioavailability ademetionine yogurt products are also prepared using one or more gallic acid esters mixed with ademetionine. In a specific embodiment, the gallic acid ester is selected from ethyl gallate, isoamyl gallate, propyl gallate and octyl gallate.

### Example 6

### Vanilla Ademetionine Yogurt Packs

Vanilla ademetionine yogurt cups for individual use are prepared using 100 mg of ademetionine. Vanilla yogurt is dispensed into 100 mL plastic yogurt cups. The cups are then heat-sealed using standard yogurt processing techniques. Stabilized ademetionine particles are dispensed into individual packets (5 cm×5 cm) such that about 100 mg of ademetionine is included in each packet. The packet is then sealed and glued with a soft, pliable glue dot onto the top of the sealed yogurt cup. A clear, plastic lid is then applied over the top of the ademetionine packet and which connects to the top of the yogurt cup such that the packet is contained, entirely underneath the plastic lid. An attractive, descriptive label is applied to each cup, along with instructions for mixing the stabilized ademetionine into the vanilla yogurt just prior to eating. Also included is the statement “Ademetionine is sensitive to moisture, best nutritional results are achieved if consumed within 30 minutes of mixing.” 12 vanilla ademetionine yogurt cups are packaged into a cardboard case which is then stored at about 4° C.

High bioavailability ademetionine yogurt cups are also prepared using one or more gallic acid esters mixed with ademetionine. In a specific embodiment, the gallic acid ester is selected from ethyl gallate, isoamyl gallate, propyl gallate and octyl gallate.

### Example 7

### Ademetionine Marble Soda

Marble pop ademetionine products are prepared using 50-3600 mg of ademetionine. A ramune or similar codd-neck bottle is filled with a carbonated liquid that is optionally flavored with cola, fruit and/or other sweet flavorings; the marble is secured above the liquid and then above the marble, stabilized ademetionine which is protected (optionally in a soluble membrane) is added such that it will not drop below the marble into the carbonated liquid until the marble is released by a topper; the topper is inserted into the bottle; the topper/bottle combination is sealed with a plastic wrapping; a label is applied to each bottle; and, optionally the bottles are packed 12 or 24 per case and then cases are stored and/or shipped prior to being sold.

Marble pop ademetionine products are designed to be sold in vending machines, food trucks, food carts, concession stands, and more including restaurants, bistros, cafes and grocery/convenience stores.

High bioavailability ademetionine marble sodas are also prepared using one or more gallic acid esters mixed with ademetionine. In a specific embodiment, the gallic acid ester is selected from ethyl gallate, isoamyl gallate, propyl gallate and octyl gallate.

### Example 8

### Mango Ademetionine Bubble Tea

Ademetionine bubble tea is prepared using 400 mg of ademetionine.

Mango-flavored tea is made in a large container. Stabilized ademetionine pearls are made by adding stabilized ademetionine granules to tapioca dough. Tapico dough is made by adding 400 grams of tapioca starch into a large mixing vessel and slowing allowing for the addition of boiling water such that upon gentle mixing a semi-solid, pliable dough is formed. The dough is allowed to cool to room temperature. Stabilized ademetionine granules are gently folded into the dough and, once fairly distributed, the dough is mechanically- or hand-separated into small balls or pearls (approximately 3 mm×3 mm). The pearls are allowed to dry and are then stored until ready to be mixed with the mango tea. Preferably the pearls are stored in airtight containers inside a refrigerator; however, they may also be stored at room temperature.

Mango ademetionine bubble tea is made by dispensing 300 mL of mango tea into a drinking container and adding approximately 40-60 dry ademetionine tapioca pearls. It is recommended to let the pearls absorb a small amount of tea so as to soften prior to drinking. For best results, tea is consumed within 20-30 minutes so as to minimize any potential ademetionine degradation and/or racemization. The mango tea is preferably not boiling hot when added to the ademetionine tapioca pearls.

High bioavailability ademetionine bubble teas are also prepared using one or more gallic acid esters mixed with ademetionine. In a specific embodiment, the gallic acid ester is selected from ethyl gallate, isoamyl gallate, propyl gallate and octyl gallate.

### Example 9

### Propyl Gallate Significantly Increases Plasma Levels of Ademetionine

Compositions comprising ademetionine and various alkyl gallates of different chemical structure were generated by mixing ademetionine with either methyl gallate, ethyl gallate, butyl gallate, isobutyl gallate, isoamyl gallate or octyl gallate (see structures below). Formulations were then fed to Beagle dogs and blood samples were withdrawn over time. Analysis of concentrations of ademetionine in plasma was used to compare the effects of these gallic acid esters on the uptake of orally administered ademetionine in the blood.

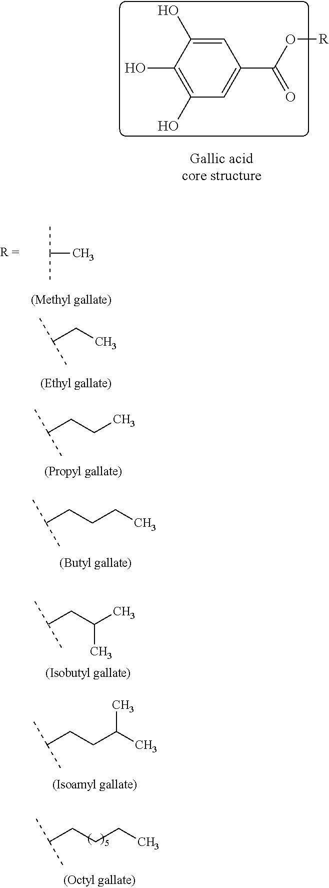

Formulations were generated by mixing 400 mg ademetionine ion (from S-adenosyl methionine disulfate tosylate) with 25 mg gallic acid ester along with excipients (microcrystalline cellulose, sodium starch glycolate, silicon dioxide, and magnesium stearate) to make up the ˜1025 mg oral formulation. A commercial seal coat and then a commercial enteric coat was applied to the formulations prior to dissolution testing. Detailed dissolution profiling was performed according to USP standards at either pH 6.8 or 6.0 on each set of formulations to ensure that adequate dissolution was achieved prior to in vivo pharmacokinetic analysis.

For in vivo studies, fasted male beagle dogs (7-10 kg) were used. The study protocol was approved by the institution's Animal Care Committee, and all animals were cared for according to regulations proposed by Agriculture Canada and the USDA. Each group (consisting of 6 dogs) was fed a single orally administered enteric coated formulation, under fasted conditions, followed by 5 mL of purified water orally with a syringe to facilitate swallowing. Blood samples (2 mL each) for ademetionine analysis were collected from the jugular vein using the following time points: pre-dose, 20 and 40 minutes, 1, 1.5, 2, 3, 4, 6, and 8 hours after treatment. The venipuncture blood samples were collected into tubes containing the anticoagulant K2-EDTA, and stored on wet ice pending processing. Following collection, samples were centrifuged (at 4° C.) to separate the plasma fraction from the blood cells. The resulting plasma fraction was recovered and stored frozen (at −80° C.) using polypropylene tubes pending bioanalytical analysis.

The concentration of ademetionine in dog plasma was determined using a well established liquid chromatography-tandem mass spectrometry (LC/MS/MS) method. This method employs stable-isotope dilution liquid chromatography-electrospray injection tandem mass spectrometry (LC-ESI-MS/MS) to determine ademetionine and SAH in plasma. The analysis used to calculate the main pharmacokinetic parameters (Cmax, Tmax and AUC) was conducted using GraphPad Prism® 5 software.

Ten different formulations were tested in the present study. 400 mg ademetionine co-formulated with 25 mg propyl gallate was compared to: ademetionine control formulations (i.e., with no propyl gallate); ademetionine formulations co-administered with separate 25 mg propyl gallate formulations; a commercially available ademetionine product (400 mg); or 400 mg ademetionine with 25 mg of either methyl gallate, ethyl gallate, butyl gallate, isobutyl gallate, isoamyl gallate or octyl gallate.

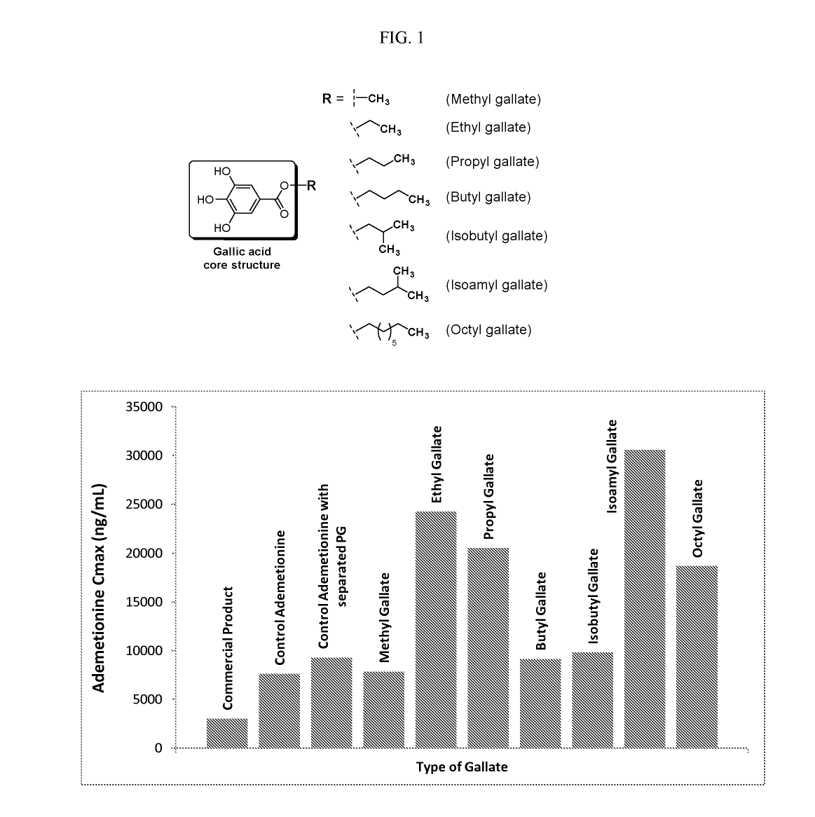

The graph in FIG. 1 clearly shows the superior combination of ademetionine and ethyl gallate, propyl gallate, isoamyl gallate or octyl gallate. The maximum ademetionine plasma concentration of these 10 formulations identifies for the first time that ademetionine co-formulated with either ethyl gallate, propyl gallate, isoamyl gallate or octyl gallate has superior uptake into the plasma as compared to the other gallic acid ester formulations tested. Surprisingly, administration of ademetionine with alkyl gallates whose alkyl moiety differs by as little as one carbon (e.g. butyl gallate) did not result in pharmacokinetics even close to that of ademetionine-ethyl gallate, propyl gallate, isoamyl gallate or octyl gallate formulations.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A drinkable or edible composition comprising ademetionine.

2. The composition of claim 1, wherein said composition comprises a milkshake, smoothie, slushie, bubble tea, pop, soda, crystal, powder, sprinkle, slurry, suspension, other instant particulate oral dosage form, snack bar, protein bar, meal-replacement bars, supplement bar, wafer, cracker, cookie, soup, cereal, cereal bar, cereal cluster, cake, yogurt, baked good, gummy, chewy product, confectionary product, sprinkle or granola.

3. The composition of claim 1, which comprises ademetionine particles wherein at least about 30%, 40%, 50%, 60%, 70%, 80%, 90% or greater than 90% of said particles are from about 10 to about 5000 microns.

4. The composition of claim 3, wherein said particle size is measured by light scattering or dynamic light scattering.

5. The composition of claim 1, wherein said ademetionine is solubilized or in solution.

6. The composition of claim 1, which further comprises at least one gallic acid ester.

7. The composition of claim 6, wherein said at least one gallic acid ester is selected from methyl gallate, ethyl gallate, propyl gallate, butyl gallate, isobutyl gallate, isoamyl gallate, octyl gallate, dodecyl gallate, and hexadecyl gallate.

8. A process for producing a shelf-ready ademetionine milkshake composition comprising:
(a) preparing a milkshake mixture comprising a milk component and a starch component;
(b) pasteurizing the mixture to form a pasteurized mixture;
(c) homogenizing the mixture to form a homogenized, pasteurized mixture;
(d) adding stabilized ademetionine to the homogenized, pasteurized mixture; and,
(e) dispensing the pasteurized, homogenized mixture into containers which are then sterilized.

9. A process for producing a shelf-ready ademetionine drink crystal or powder composition comprising:
(a) preparing a mixture comprising ademetionine, an antifoaming agent and a flavorant;
(b) combining and then stirring the above mixture with sugar;
(c) drying the resulting slurry; and
(d) grinding the obtained dried crystals to a desired size crystal or powder.

10. The composition of claim 6, wherein the ratio (weight:weight) of gallic acid ester to ademetionine is from 5:1 to 1:400 or 1:1 to 1:100.

11. The composition of claim 1, wherein the composition when administered to a selected subject group provides in said selected subject group an average maximum ademetionine blood plasma concentration (average Cₘₐₓ) of at least about 1.2 ng/mL and/or an average ademetionine plasma area under the curve (average AUC) of at least about 8 ng·h/mL per each 1 mg dosage of ademetionine ion.

12. The composition of claim 1, which comprises from about 10 mg to about 3600 mg of ademetionine.

13. The composition of claim 1, which comprises at least 5%, at least 10%, at least 15%, at least 20%, at least 25%, at least 30%, at least 35%, at least 40%, at least 45%, at least 50%, or at least greater than 50% by weight ademetionine ion.

14. A method of treating a disease or disorder selected from the group consisting of a mental or psychiatric disorder, nervous system disease or disorder, neurological disease or disorder, condition associated with injury to the central nervous system, liver disease or disorder, cancer, joint disease or disorder, inflammatory disease or disorder, autoimmune disease or disorder, degenerative disease or disorder, soft-tissue disease or disorder, pain disease or disorder, genetic disorder related to hyper- or hypo-methylation, gastrointestinal disease or disorder, cardiovascular disease or disorder, atherosclerosis, Lesch-Nyhan disease, and disorder induced in whole or in part by oxidative or free-radical damage comprising administering a composition of claim 1 to a subject in need thereof.

15. A method of increasing the rate of onset of treatment of a disease or disorder selected from the group consisting of a mental or psychiatric disorder, nervous system disease or disorder, neurological disease or disorder, condition associated with injury to the central nervous system, liver disease or disorder, cancer, joint disease or disorder, inflammatory disease or disorder, autoimmune disease or disorder, degenerative disease or disorder, soft-tissue disease or disorder, pain disease or disorder, genetic disorder related to hyper- or hypo-methylation, gastrointestinal disease or disorder, cardiovascular disease or disorder, atherosclerosis, Lesch-Nyhan disease, and disorder induced in whole or in part by oxidative or free-radical damage comprising administering a composition of claim 1 to a subject in need thereof.

16. The method of claim 14 or 15, wherein the mental or psychiatric disorder is selected from the group consisting of an anxiety disorder, schizophrenia, major depressive disorder, major depression, clinical depression, dysthymic disorder, anxiety depression, atypical depression, melancholic depression, catatonic depression, situational depression, reactive depression, late-life depression, Seasonal Affective Disorder (SAD), minor depression, postpartum depression, inflammatory depression, late-life depression, brief recurrent depression, mild depression, treatment-resistant depression (TRD), co-morbid depression, Parkinson's depression, HIV-associated depression, multi-infarct dementia, and bipolar disorder;
the inflammatory disease or disorder is selected from the group consisting of systemic lupus, inflammatory bowel disease, allergic rhinitis, contact dermatitis, asthma, autoimmune hepatitis, and pelvic inflammatory disease;
the cardiovascular disease or disorder is selected from the group consisting of hyper- or hypo-homocysteinemia, coronary heart disease, stroke, peripheral vascular disease, and atherosclerotic disease;
the depressive disorder is a co-morbid depression arising in a subject who is or has been undergoing treatment for one or more diseases or disorders selected from the group consisting of cancer, Parkinson's disease, and HIV;
the nervous system disease or disorder or injury is selected from the group consisting of Parkinson's disease, Alzheimer's disease, and cognitive impairment;
the liver disease or disorder is selected from the group consisting of alcoholic liver disease, non-alcoholic fatty liver disease, viral or non-viral hepatitis, liver cancer, oxidative liver disease, drug induced liver injury, cholestasis, and cirrhosis;
the cancer is selected from the group consisting of liver cancer, bowel cancer, colon cancer, rectal cancer, colorectal cancer, stomach cancer, esophageal cancer, and adenocarcinoma;
the joint disease or disorder is arthritis or osteoarthritis;
the soft-tissue disease or disorder is fibromyalgia;
the pain disease or disorder is fibromyalgia or abdominal pain; or
the genetic disorder related to hyper- or hypo-methylation is methylenetetrahydrofolate reductase deficiency.

17. A method of making a composition for improved appealability of ademetionine, wherein said method comprises formulating ademetionine into a drinkable or edible dosage form for human consumption.

18. A method for improving the appealability of ademetionine, wherein said method comprises administering to a human subject an exemplary drinkable or edible composition which provides a physiologically effective amount of ademetionine.

19. A method for ingesting ademetionine, wherein said method comprises administering to a human subject a drinkable or edible composition which provides a physiologically effective amount of ademetionine.

20. The method of any one of claims 17-19, wherein said drinkable or edible composition comprises at least one gallic acid ester.

21. The composition of claim 1, wherein said composition does not comprise silybin, dissolved oxygen, ten or more amino acids, ten or more enzymes and/or one or more aerobic proteins.

22. The composition of claim 1, wherein said composition is a gummy or chewy product (including a carbohydrate and/or energy chew and/or gel), a confectionary product (including bakers' confections and sugar confections including candies, pastilles and sweets).

23. The composition of claim 1, which is not a tablet or capsule, including for example, minitablets, orodispersible tablets, soft-gel capsules and hard-gel capsules.
